(19) United States
(12) Patent Application Publication
(10) Pub. No.: US 2015/0202065 A1
(43) Pub. Date: Jul. 23, 2015

(54) STENT-GRAFTS CONFIGURED FOR POST-IMPLANTATION EXPANSION

(71) Applicant: ENDOSPAN LTD., Herzilyia Pituach (IL)

(72) Inventors: Alon Shalev, Ra'anana (IL); Nir Shalom Nae, Ra'anana (IL)

(73) Assignee: ENDOSPAN LTD., Herzilyia Pituach (IL)

(21) Appl. No.: 14/416,236

(22) PCT Filed: Jul. 31, 2013
(86) PCT No.: PCT/IL13/50656

§ 371 Date: Jan. 21, 2015

Related U.S. Application Data
(60) Provisional application No. 61/678,182, filed on Aug. 1, 2012.

(51) Int. Cl. A61F 2/93

(52) U.S. Cl. CPC A61F 2/93

(57) ABSTRACT

An endovascular stent-graft is provided that includes a generally tubular body, which (a) is configured to assume a radially-compressed delivery state and at least first and second radially-expanded deployment states, (b) is shaped so as to define a stepwise expanding portion, and (c) comprises a stent member. The stent member includes a plurality of self-expandable flexible structural stent elements, and at least one circumferential expansion element. The stent member is configured such that application of a force thereto, which is insufficient to cause plastic deformation of the self-expandable flexible structural stent elements and is sufficient to cause plastic deformation of the circumferential expansion element, causes an increase in a circumferential length of the circumferential expansion element, thereby transitioning the tubular body from the first radially-expanded deployment state to the second radially-expanded deployment state, thereby increasing a greatest internal perimeter of the expanding portion.

### CROSS-REFERENCE TO RELATED APPLICATIONS

The present application is the U.S. national stage of International Application PCT/IL2013/050656, filed Jul. 31, 2013, which claims priority from U.S. Provisional Application 61/678,182, filed Aug. 1, 2012, which is assigned to the assignee of the present application and is incorporated herein by reference.

### FIELD OF THE APPLICATION

The present application relates generally to prostheses and surgical methods, and specifically to tubular prostheses, including endovascular stent-grafts, and surgical techniques for using the prostheses to maintain patency of body passages such as blood vessels, and treating aneurysms and dissections of arterial walls.

### BACKGROUND OF THE APPLICATION

An aneurysm is a localized, blood-filled dilation (bulge) of a blood vessel caused by disease or weakening of the vessel wall. Left untreated, the aneurysm will frequently rupture, resulting in loss of blood through the rupture and death. Endovascular prostheses are sometimes used to treat aortic aneurysms. Such treatment includes implanting a stent or stent-graft within the diseased vessel to bypass the anomaly. Aneurysms may be congenital, but are usually caused by disease or, occasionally, by trauma. Aortic aneurysms include abdominal aortic aneurysms (“AAAs”), which form between the renal arteries and the iliac arteries, and thoracic aortic aneurysms (“TAAs”), which may occur in one or more of the descending aorta, the ascending aorta, and the aortic arch.

“Endoleak” is the persistent flow of blood into the aneurysm sac after implantation of an endovascular prosthesis. The management of some types of endoleak remains controversial, although most can be successfully occluded with surgery, further stent implantation, or embolization. Four types of endoleaks have been defined, based upon their proposed etiology.

A type I endoleak, which occurs in up to 10 percent of endovascular aortic aneurysm repairs, is due to an incompetent seal at either the proximal or distal attachment sites of the vascular prosthesis, resulting in blood flow at the end of the prosthesis into the aneurysm sac. Etiologies include undersizing of the diameter of the endograft at the attachment site and ineffective attachment to a vessel wall that is heavily calcified or surrounded by thick thrombus. Type I failures have also been found to be caused by a continual expansion of the aneurysm neck (the portion of the aorta extending cephalad or caudad from the aneurysm, which is not dilated). This expansion rate has been estimated to be about one millimeter per year. Because the aneurysm neck expands beyond the natural resting diameter of the prosthesis, one or more passageways are defined about the prosthesis in communication with the aneurysm sac. Additionally, type I endoleaks may be caused when circular prostheses are implanted in non-circular aortic lumens, which may be caused by irregular vessel formation and/or calcified topography of the lumen of the aorta.

Type I endoleaks may occur immediately after placement of the prosthesis, or may be delayed. A delayed type I endoleak may be seen during follow-up studies if the prosthesis is deployed into a diseased segment of aorta that dilates over time, leading to a breach in the seal at the attachment site.

Type I endoleaks must be repaired as soon as they are discovered, because the aneurysm sac remains exposed to systemic pressure, predisposing to aneurysmal rupture, and spontaneous closure of the leak is rare. If discovered at the time of initial placement, repair may consist of reversal of anticoagulation and reinflation of the deployment balloon for an extended period of time. These leaks may also be repaired with small extension grafts that are placed over the affected end. These methods are usually sufficient to exclude the aneurysm. Conversion to an open surgical repair may be needed in the rare situation in which the leak is refractory to percutaneous treatment.

Research has shown that the necks of the post-surgical aorta increase in size for approximately twelve months after implantation of a stent-graft, regardless of whether the aneurysm experiences dimensional change. This phenomenon can result in perigraft leaks and graft migration. Furthermore, progressive expansion of the aneurysm sac associated with type I endoleak can lead to compromise of the seal at the neck and is the principal indication for secondary intervention for this condition.

Sizing of aortic endografts is an essential step in successful endovascular treatment of aortic pathology, although there is no consensus regarding the optimal sizing strategy. Some proximal oversizing is necessary to obtain a seal between the stent-graft and the aortic wall and to prevent the graft from migrating, but excessive oversizing might negatively influence the results. In a systematic review, the current literature was investigated to obtain an overview of the risks and benefits of oversizing and to determine the optimal degree of oversizing of stent-grafts used for endovascular abdominal aortic aneurysm repair (J van Prehn et al., “Oversizing of Aortic Stent Grafts for Abdominal Aneurysm Repair: A Systematic Review of the Benefits and Risks,” European Journal of Vascular & Endovascular Surgery 38(1):42-53, July 2009 (published online May 11, 2009)). Prehn et al. conclude that “based on the best available evidence, the current standard of 10-20% oversizing regime appears to be relatively safe and preferable. Oversizing >30% might negatively impact the outcome after EVAR. Studies of higher quality are needed to further assess the relationship between proximal oversizing and the incidence of complications, particularly regarding the impact on aneurysm neck dilatation.”

In light of the above, it appears that the functional lifespan of a stent-graft is limited, because (a) the pathology is progressive and (b) there is an upper limit on desirable oversizing, which if crossed, may itself exacerbate the proximal neck expansion rate and hence contribute to type I endoleak and device migration.

PCT Publication WO 2009/078010 to Shalev, and US Patent Application Publication 2010/0292774 in the national stage thereof, which are assigned to the assignee of the present application and are incorporated herein by reference, describe a system for treating an aneurysmatic abdominal aorta, comprising (a) an extra-vascular wrapping (EVW) comprising (i) at least one medical textile member adapted to at least partially encircle a segment of aorta in proximity to the renal arteries, and (ii) a structural member, wherein the EVW is adapted for laparoscopic delivery, and (b) an endovascular stent-graft (ESG) comprising (i) a compressible structural member, and (ii) a substantially fluid impervious fluid flow guide (FFG) attached thereto. Also described is an extra-vascular ring (EVR) adapted to encircle the neck of an aortic aneurysm. Further described are methods for treating an abdominal aortic aneurysm, comprising laparoscopically delivering the extra-vascular wrapping (EVW) and endovascularly placing an endovascular stent-graft (ESG). Also described are methods to treat a type I endoleak. U.S. Provisional Application 61/014,031, filed Dec. 15, 2007, from which the above-referenced applications claim priority, is also incorporated herein by reference.

### SUMMARY OF APPLICATIONS

Applications of the present invention provide endovascular stent-grafts that are configured to be radially expanded during minimally-invasive secondary intervention procedures performed after completion of implantation of the stent-grafts, typically upon detection of type I endoleak or concern of migration. The stent-grafts of the present invention thus minimize the invasiveness of secondary endovascular intervention, and provide techniques that can easily and safely be performed by a surgeon or interventionalist that is skilled in the art of endovascular aortic interventions. These techniques help prevent the need for more invasive and costly intervention, such as implantation of a flared, larger diameter proximal extension cuff, or surgical repair of the endoleak and/or migration.

Each of the stent-grafts of the present invention comprises a generally tubular body. The tubular body is configured to assume (a) a radially-compressed delivery state, typically when the body is initially positioned in a delivery catheter, and (b) at least first and second radially-expanded deployment states. The body typically assumes the first radially-expanded deployment state upon deployment from the delivery catheter, and the second radially-expanded delivery state after deployment, typically during a minimally-invasive secondary intervention procedure. In order to enable a transition from the first radially-expanded deployment state to the second radially-expanded deployment state, the tubular body is shaped so as to define a stepwise expanding portion, a greatest internal perimeter of which increases as the body transitions from the first radially-expanded deployment state to the second radially-expanded delivery state. The tubular body comprises a stent member, and, typically, a generally tubular fluid flow guide comprising a graft material, which is attached to the stent member. The fluid flow guide is configured to accommodate the increasing of the greatest internal perimeter of the expanding portion, as described hereinbelow.

For some applications, the stent member comprises a plurality of self-expandable flexible structural stent elements, and a circumferential expansion element that is coupled to at least two of the self-expandable flexible structural stent elements of the expanding portion of the tubular body. The structural stent elements comprise a self-expanding material, such as a self-expanding metal, such that the body is self-expandable. For some applications, the circumferential expansion element is generally non-elastic. For example, the circumferential expansion element may comprise non-elastic stainless steel, or a cobalt-chromium alloy.

The stent member is configured such that application of a force thereto, which is insufficient to cause plastic deformation of the self-expandable flexible structural stent elements and is sufficient to cause plastic deformation of the circumferential expansion element, causes an increase in a circumferential length of the circumferential expansion element. This increase in length transitions the tubular body from the first radially-expanded deployment state to the second radially-expanded deployment state, thereby increasing the greatest internal perimeter of the expanding portion.

As mentioned above, the fluid flow guide is configured to accommodate the increasing of the greatest internal perimeter of the expanding portion. For some applications, in order to provide such accommodation, when the tubular body is in the first radially-expanded deployment state, the fluid flow guide is shaped so as to define one or more folds in a vicinity of the circumferential expansion element. For some applications, when the tubular body is in the first radially-expanded deployment state, the one or more folds are disposed radially outside the stent member. Alternatively, for some applications, in order to provide such accommodation, at least a portion of the fluid flow guide in a vicinity of the circumferential expansion element comprises a stretchable fabric.

For some applications, the stent-graft comprises a circumferential expansion prevention element, which is coupled to at least two self-expandable flexible structural stent elements of the expanding portion of the tubular body. When intact, the circumferential expansion prevention element restrains the tubular body in the first radially-expanded deployment state, in which the expanding portion has a first greatest internal perimeter. When detached and/or severed, such as by application of a force that increases a distance between the two stent elements to which the circumferential expansion prevention element is coupled, the circumferential expansion prevention element does not restrain the tubular body in the first radially-expanded deployment state. As a result, the tubular body transitions from the first radially-expanded deployment state to the second radially-expanded deployment state. In the second radially-expanded deployment state, the expanding portion has a second greatest internal perimeter, which is greater than the first greatest internal perimeter.

For some applications, the circumferential expansion prevention element comprises a suture, a wire (e.g., comprising metal), a hook, a loop, or a helix. The circumferential expansion prevention element is detached and/or severed, such as by cutting or breaking thereof. For example, a cutting tool may be used, or a balloon may be used to apply a force sufficient to detach and/or sever the element, by increasing a distance between the two stent elements to which the element is coupled.

For some applications, the graft material of the fluid flow guide is shaped so as to define, when the tubular body is in the first radially-expanded deployment state, one or more folds disposed such that at least 50%, e.g., at least 75%, such as 100%, of the graft material of the folds is radially outside the stent member. Disposing of the folds mostly or entirely outside of the stent member reduces or prevents any interfere by the folds with the flow of blood through the fluid flow guide. If the folds instead extended mostly or entirely into the lumen of the fluid flow guide, the folds would reduce the effective cross-section of the lumen and potentially interfere with blood flow and increase the risk of thrombosis. Such interference is particularly undesirable because the stent-graft often remains implanted in the first radially-expanded deployment state for an extended period of time, such as months or years, or even permanently. For some applications, the graft material is shaped so as to define exactly one or exactly two folds when the tubular body is in the first radially-expanded deployment state.

Typically, when the tubular body is in the second radially-expanded deployment state, the graft material of the fluid flow guide is shaped so as to define none of the folds or fewer of the folds than when the tubular body is in the first radially-expanded deployment state.

For some applications, when the tubular body is in the first radially-expanded deployment state, the one or more folds are oriented tangentially to the tubular body, such that a portion of the graft material of the one or more folds is in contact with an outer surface of the tubular body.

For some applications, each of the one or more folds is relatively large with respect to the greatest internal perimeter of the expanding portion, in order to provide a large circumferential buffer for expansion of the expanding portion after implantation. For example, a greatest internal perimeter of the graft material of a first one of the one or more folds, when the first fold is unfolded when the tubular body is in the second radially-expanded deployment state, may be equal to at least 7% of the second greatest internal perimeter.

For some applications, a locking mechanism is provided, which is configured to assume a locked state which restrains the tubular body in the first radially-expanded deployment state, and a released state, which allows the tubular body to transition to the second radially-expanded deployment state.

For some applications, during a primary intervention procedure, a surgeon or interventionalist transvascularly (e.g., transcutaneously) introduces the stent-graft into a blood vessel while the tubular body of the stent-graft is in the radially-compressed delivery state. Thereafter, the surgeon or interventionalist transitions the tubular body to the first radially-expanded deployment state in the blood vessel, in which state the expanding portion has the first greatest internal perimeter and forms a blood-tight seal with a wall of the blood vessel at a neck of an aneurysm and/or a dissection of an arterial wall. The initial implantation procedure is complete.

Over time (typically over a few years), the neck of the aneurysm often progressively dilates, such as because of progressive expansion of the aneurysm sac. Such dilation of the neck may compromise the seal between the expanding portion of the stent-graft and the wall of the anatomical neck, resulting in type I endoleak. In response to detecting such dilation and/or endoleak (typically at least one month, such as at least one year, e.g., a few years, after initial implantation and deployment of the stent-graft), a surgeon or interventionalist, during a minimally-invasive secondary intervention procedure, transitions the tubular body to the second radially-expanded deployment state in the blood vessel. In the second radially-expanded deployment state, the expanding portion has the second greatest internal perimeter, which is greater than the first greatest perimeter.

There is therefore provided, in accordance with an application of the present invention, apparatus including an endovascular stent-graft system, which includes an endovascular stent-graft, which includes a generally tubular body, which:
is shaped so as to define a stepwise expanding portion,
is configured to assume (a) a radially-compressed delivery state and (b) at least first and second radially-expanded deployment states, in which the expanding portion has respective first and second greatest internal perimeters, the second greater than the first, wherein the tubular body, when in the first radially-expanded deployment state, is restrained from transitioning to the second radially-expanded deployment state, and
includes a self-expandable flexible stent member, and a generally tubular fluid flow guide, which is attached to the stent member and includes a graft material that is shaped so as to define, when the tubular body is in the first radially-expanded deployment state, one or more folds disposed such that at least 50% of the graft material of the folds is radially outside the stent member.

For some applications, at least 75%, such as 100%, of the graft material of the folds is radially outside the stent member when the tubular body is in the first radially-expanded deployment state.

For some applications, when the tubular body is in the second radially-expanded deployment state, the graft material of the fluid flow guide is shaped so as to define none of the folds or fewer of the folds than when the tubular body is in the first radially-expanded deployment state.

For some applications, the second greatest internal perimeter of the expanding portion is at least 10% greater than the first greatest internal perimeter of the expanding portion.

For some applications, when the tubular body is in the first radially-expanded deployment state, the one or more folds are oriented tangentially to the tubular body, such that a portion of the graft material of the one or more folds is in contact with an outer surface of the tubular body. For some applications, wherein, at least when the tubular body is in the radially-compressed delivery state, the one or more folds are removably secured to the outer surface of the tubular body. For some applications, the apparatus further includes a securing mechanism, which removably secures the folds to the outer surface of the tubular body. For some applications, the apparatus further includes a biodegradable adhesive, which removably secures the folds to the outer surface of the tubular body.

For some applications, the expanding portion is disposed at a longitudinal end of the body.

For some applications, a greatest internal perimeter of the graft material of a first one of the one or more folds, when the first fold is unfolded when the tubular body is in the second radially-expanded deployment state, is at least 7% of the second greatest internal perimeter. Alternatively or additionally, for some applications, a greatest internal perimeter of the graft material of a second one of the one or more folds, when the second fold is unfolded, is at least 7% of the second greatest internal perimeter.

For some applications, the stent-graft system further includes a locking mechanism, configured to assume a locked state which restrains the tubular body in the first radially-expanded deployment state, and a released state, which allows the tubular body to transition to the second radially-expanded deployment state. For some applications, the locking mechanism includes a shaft and two or more attachment members coupled to the stent-graft, the shaft passes through the attachment members when the locking mechanism is in the locked state, and the shaft does not pass through the attachment members when the locking mechanism is in the released state. For some applications, the locking mechanism transitions from the locked state to the released state in response to translation of the shaft. For some applications, the translation is longitudinal translation.

For some applications, one of the one or more folds has two end portions at a surface generally defined by the tubular body, and, when the stent-graft is in the first radially-expanded deployment state, a length of the fold, measured along the graft material of the fold at a longitudinal end of the body between the two end portions, is at least 140% of a distance between the two end portions of the fold at the longitudinal end. For some applications, when the stent-graft is in the first radially-expanded deployment state, the length of the fold, measured along the graft material of the fold at the longitudinal end of the body between the two end portions, is at least 167% of the distance between the two end portions of the fold at the longitudinal end. For some applications, when the stent-graft is in the first radially-expanded deployment state, the length of the fold, measured along the graft material of the fold at the longitudinal end of the body between the two end portions, is at least 500% of the distance between the two end portions of the fold at the longitudinal end.

For some applications, the graft material is shaped so as to define exactly one or exactly two folds when the tubular body is in the first radially-expanded deployment state.

For some applications, the stent member includes:
a plurality of self-expandable flexible structural stent elements; and
a circumferential expansion element that is coupled to at least two of the self-expandable flexible structural stent elements of the expanding portion of the tubular body, and
the stent member is configured such that application of a force thereto, which is insufficient to cause plastic deformation of the self-expandable flexible structural stent elements and is sufficient to cause plastic deformation of the circumferential expansion element, causes an increase in a circumferential length of the circumferential expansion element, thereby transitioning the tubular body from the first radially-expanded deployment state to the second radially-expanded deployment state.

There is further provided, in accordance with an application of the present invention, apparatus including an endovascular stent-graft system, which includes a generally tubular body, which:
is shaped so as to define a stepwise expanding portion,
is configured to assume (a) a radially-compressed delivery state and (b) at least first and second radially-expanded deployment states, in which the expanding portion has respective first and second greatest internal perimeters, the second greater than the first, wherein the tubular body, when in the first radially-expanded deployment state, is restrained from transitioning to the second radially-expanded deployment state, and
includes a self-expandable flexible stent member, and a generally tubular fluid flow guide, which is attached to the stent member and includes a graft material that is shaped so as to define, when the tubular body is in the first radially-expanded deployment state, one or more folds,
wherein a greatest internal perimeter of the graft material of a first one of the one or more folds, when the first fold is unfolded when the tubular body is in the second radially-expanded deployment state, is at least 7% of the second greatest internal perimeter.

For some applications, the first length equals at least 10% of the second greatest internal perimeter.

For some applications, a greatest internal perimeter of the graft material of a second one of the one or more folds, when the second fold is unfolded, is at least 7% of the second greatest internal perimeter.

For some applications, when the tubular body is in the second radially-expanded deployment state, the fluid flow guide is shaped so as to define none of the folds or fewer of the folds than when the tubular body is in the first radially-expanded deployment state.

For some applications, the second greatest internal perimeter is at least 10% greater than the first greatest internal perimeter.

For some applications, the stent-graft system further includes a locking mechanism, configured to assume a locked state which restrains the tubular body in the first radially-expanded deployment state, and a released state, which allows the tubular body to transition to the second radially-expanded deployment state.

There is still further provided, in accordance with an application of the present invention, apparatus including an endovascular stent-graft system, which includes a generally tubular body, which:
is shaped so as to define a stepwise expanding portion,
is configured to assume (a) a radially-compressed delivery state and (b) at least first and second radially-expanded deployment states, in which the expanding portion has respective first and second greatest internal perimeters, the second greater than the first, wherein the tubular body, when in the first radially-expanded deployment state, is restrained from transitioning to the second radially-expanded deployment state, and
includes a self-expandable flexible stent member, and a generally tubular fluid flow guide, which is attached to the stent member and includes a graft material that is shaped so as to define, when the tubular body is in the first radially-expanded deployment state, exactly one or exactly two folds.

For some applications, the graft material that is shaped so as to define exactly one fold when the tubular body is in the first radially-expanded deployment state.

There is additionally provided, in accordance with an application of the present invention, apparatus including an endovascular stent-graft, which includes a generally tubular body, which tubular body (a) is configured to assume a radially-compressed delivery state and at least first and second radially-expanded deployment states, (b) is shaped so as to define a stepwise expanding portion, and (c) includes a stent member, which includes:
a plurality of self-expandable flexible structural stent elements; and
at least one circumferential expansion element,
wherein the stent member is configured such that application of a force thereto, which is insufficient to cause plastic deformation of the self-expandable flexible structural stent elements and is sufficient to cause plastic deformation of the circumferential expansion element, causes an increase in a circumferential length of the circumferential expansion element, thereby transitioning the tubular body from the first radially-expanded deployment state to the second radially-expanded deployment state, thereby increasing a greatest internal perimeter of the expanding portion.

For some applications, the circumferential expansion element circumscribes an angle of at least 3 degrees, e.g., at least 5 degrees, when the tubular body is in the first radially-expanded deployment state.

For some applications, the circumferential expansion element is coupled to at least two of the self-expandable flexible structural stent elements of the expanding portion of the tubular body. For some applications, a pair of the at least two of the self-expandable flexible structural stent elements to which the circumferential expansion element is coupled are coupled at a peak.

For some applications, the self-expandable flexible structural stent elements of the stent member are shaped so to define at least one circumferential band at the expanding portion, which band is shaped so as to define a plurality of peaks directed in a first longitudinal direction, alternating with a plurality of troughs directed in a second longitudinal direction opposite the first longitudinal direction. For some applications, the at least one circumferential expansion element is positioned alongside one of the self-expandable flexible structural stent elements near an element selected from the group consisting of: one of the peaks and one of the troughs. For some applications, the at least one circumferential expansion element is shaped similarly to a portion of the self-expandable flexible structural stent elements alongside which the at least one circumferential expansion element is positioned.

For some applications, the tubular body further includes a generally tubular fluid flow guide, which (a) includes a graft material, (b) is attached to the stent member, and (c) is configured to accommodate the increasing of the greatest internal perimeter of the expanding portion. For some applications, the at least one circumferential expansion element is attached to the fluid flow guide. For some applications, when the tubular body is in the first radially-expanded deployment state, the fluid flow guide is shaped so as to define one or more folds in a vicinity of the circumferential expansion element, so as to accommodate the increasing of the greatest internal perimeter of the expanding portion. For some applications, when the tubular body is in the first radially-expanded deployment state, the one or more folds are disposed radially outside the stent member.

For some applications, at least a portion of the fluid flow guide in a vicinity of the circumferential expansion element includes a stretchable fabric, so as to accommodate the increasing of the greatest internal perimeter of the expanding portion. For some applications, the fluid flow guide, other than the portion in the vicinity of the circumferential expansion element, includes a fabric that is less elastic than the stretchable fabric.

For some applications, a resistance of the fluid flow guide to lateral expansion is less than 70% of a resistance of the circumferential expansion element to lateral expansion, when the tubular body is in the second radially-expanded deployment state. For some applications, the resistance of the fluid flow guide to lateral expansion is less than 30% of the resistance of the circumferential expansion element to lateral expansion, when the tubular body is in the second radially-expanded deployment state.

For some applications, the circumferential expansion element has a shape selected from the group of shapes consisting of: a U-shape, a V-shape, a W-shape, and an undulating shape, at least when the tubular body is in the first radially-expanded deployment state.

For some applications, the apparatus further includes one or more balloons, configured to apply the force from within the tubular body. For some applications, the one or more balloons include a plurality of balloons have respective different volumes when inflated.

For some applications, the circumferential expansion element includes non-elastic stainless steel.

For some applications, the circumferential expansion element is generally non-elastic. For some applications, an angular segment of the expanding portion that includes the circumferential expansion element expands and contracts at least 50% less per unit circumferential arc angle than an angular segment of the expanding portion that does not include the circumferential expansion element, as the body cycles between being internally pressurized by (a) fluid having a pressure of 80 mmHg and (b) fluid having a pressure of 120 mmHg.

For some applications, the circumferential expansion element includes a cobalt-chromium alloy.

There is yet additionally provided, in accordance with an application of the present invention, apparatus including an endovascular stent-graft, which includes a generally tubular body, which tubular body (a) is configured to assume a radially-compressed delivery state and at least first and second radially-expanded deployment states, (b) is shaped so as to define a stepwise expanding portion, and (c) includes:
a stent member, which includes a plurality of self-expandable flexible structural stent elements, which, when unconstrained, are configured to cause the tubular body to assume the second radially-expanded deployment state; and
a circumferential expansion prevention element, which is coupled to at least two of the self-expandable flexible structural stent elements of the expanding portion of the tubular body,
wherein, when intact, the circumferential expansion prevention element restrains the tubular body in the first radially-expanded deployment state, in which the expanding portion has a first greatest internal perimeter, and
wherein, when detached or severed, the circumferential expansion prevention element does not restrain the tubular body in the first radially-expanded deployment state, such that the tubular body transitions from the first radially-expanded deployment state to the second radially-expanded deployment state, in which the expanding portion has a second greatest internal perimeter, which is greater than the first greatest internal perimeter.

For some applications, the tubular body further includes a generally tubular fluid flow guide, which includes a graft material and is attached to the stent member, and is configured to accommodate the increasing of the greatest internal perimeter of the expanding portion during the transitioning.

For some applications, the circumferential expansion prevention element includes an element selected from the group consisting of: a suture, a wire, a hook, a loop, and a helix.

For some applications, the circumferential expansion prevention element circumscribes an angle of at least 3 degrees, e.g., at least 5 degrees, when the tubular body is in the first radially-expanded deployment state.

For some applications, the self-expandable flexible structural stent elements of the stent member are shaped so to define at least one circumferential band at the expanding portion, which band is shaped so as to define a plurality of peaks directed in a first longitudinal direction, alternating with a plurality of troughs directed in a second longitudinal direction opposite the first longitudinal direction; and the circumferential expansion prevention element is coupled to the at least two of the structural elements within 30% of a diameter of the body in its first radially-expanded state of respective ones of the peaks. For some applications, the circumferential expansion prevention element is coupled to the at least two of the structural elements at respective ones of the peaks.

For some applications, when the tubular body is in the first radially-expanded deployment state, the fluid flow guide is shaped so as to define one or more folds in a vicinity of the circumferential expansion prevention element, so as to accommodate the increasing of the greatest internal perimeter of the expanding portion. For some applications, when the tubular body is in the first radially-expanded deployment state, the one or more folds are disposed radially outside the stent member.

For some applications, at least a portion of the fluid flow guide in a vicinity of the circumferential expansion prevention element includes a stretchable fabric, so as to accommodate the increasing of the greatest internal perimeter of the expanding portion. For some applications, the fluid flow guide, other than the portion in the vicinity of the circumferential expansion prevention element, includes a fabric that is less elastic than the stretchable fabric.

For some applications, the apparatus further includes one or more balloons, configured to apply, from within the tubular body, a force sufficient to sever the circumferential expansion prevention element. For some applications, the one or more balloons include a plurality of balloons have respective different volumes when inflated.

There is also provided, in accordance with an application of the present invention, a method including:
providing an endovascular stent-graft, which includes a generally tubular body, which (a) is shaped so as to define a stepwise expanding portion, and (b) includes a self-expandable flexible stent member, and a generally tubular fluid flow guide, which includes a graft material and is attached to the stent member;
during a minimally-invasive primary intervention procedure, transvascularly introducing the stent-graft into a blood vessel of a human subject while the tubular body of the stent-graft is in a radially-compressed delivery state, and, thereafter, transitioning the tubular body to a first radially-expanded deployment state in the blood vessel, in which state the expanding portion has a first greatest internal perimeter and forms a blood-tight seal with a wall of the blood vessel; and
thereafter, during a minimally-invasive secondary intervention procedure separate from the primary intervention procedure, transitioning the tubular body to a second radially-expanded deployment state in the blood vessel, in which state the expanding portion has a second greatest internal perimeter and forms a blood-tight seal with the wall of the blood vessel, which second greatest internal perimeter is greater than the first greatest internal perimeter.

For some applications, transitioning the tubular body to the second radially-expanded deployment state in the blood vessel includes performing the secondary intervention procedure at least one month after performing the primary intervention procedure.

For some applications, the minimally-invasive secondary intervention procedure is a transvascular secondary intervention procedure, and transitioning the tubular body to the second radially-expanded deployment state includes transitioning the tubular body to the second radially-expanded deployment state during the transvascular secondary intervention procedure. For some applications, transitioning the tubular body to the second radially-expanded deployment state in the blood vessel includes transvascularly introducing a balloon into the tubular body, and inflating the balloon.

For some applications, the method further includes, after the minimally-invasive secondary intervention procedure, during a minimally-invasive tertiary intervention procedure separate from the primary and the secondary intervention procedures, transitioning the tubular body to a third radially-expanded deployment state in the blood vessel, in which state the expanding portion has a third greatest internal perimeter and forms a blood-tight seal with the wall of the blood vessel, which third greatest internal perimeter is greater than the second greatest internal perimeter.

For some applications, the method further includes, after transitioning the tubular body to the first radially-expanded deployment state, detecting type I endoleak, and transitioning the tubular body to the second radially-expanded deployment state includes transitioning the tubular body to the second radially-expanded deployment state in response to detecting the type I endoleak.

For some applications, the method further includes identifying that the blood vessel has an aneurysm, transitioning the tubular body to the first radially-expanded deployment state includes transitioning the tubular body to the first radially-expanded deployment state so that the expanding portion forms the blood-tight seal with the wall of the blood vessel at a neck of the aneurysm, and transitioning the tubular body to the second radially-expanded deployment state includes transitioning the tubular body to the second radially-expanded deployment state so that the expanding portion forms the blood-tight seal with the wall of the blood vessel at the neck of the aneurysm.

For some applications, transitioning the tubular body to the second radially-expanded deployment state includes transitioning the tubular body to the second radially-expanded deployment state such that the second greatest internal perimeter of the expanding portion is at least 10% greater than the first greatest internal perimeter of the expanding portion.

For some applications, providing the endovascular stent-graft includes providing the endovascular stent-graft in which the expanding portion is disposed at a longitudinal end of the body.

For some applications, transvascularly introducing the stent-graft includes transvascularly introducing the stent-graft into the blood vessel while a locking mechanism is in a locked state which restrains the tubular body in the first radially-expanded deployment state, and transitioning the tubular body to the second radially-expanded deployment state includes transitioning the locking mechanism to a released state, which allows the tubular body to transition to the second radially-expanded deployment state.

For some applications, transitioning the tubular body to the first radially-expanded deployment state includes transitioning the tubular body to the first radially-expanded deployment state such that the graft material is shaped so as to define one or more folds disposed such that at least 50% of the graft material of the folds is radially outside the stent member. For some applications, transitioning the tubular body to the first radially-expanded deployment state includes transitioning the tubular body to the first radially-expanded deployment state such that the graft material is shaped so as to define one or more folds disposed such that at least 75%, such as 100%, of the graft material of the folds is radially outside the stent member.

For some applications, transitioning the tubular body to the second radially-expanded deployment state includes transitioning the tubular body to the second radially-expanded deployment state such that the graft material of the fluid flow guide is shaped so as to define none of the folds or fewer of the folds than when the tubular body is in the first radially-expanded deployment state.

For some applications, transitioning the tubular body to the first radially-expanded deployment state includes transitioning the tubular body to the first radially-expanded deployment state such that the one or more folds are oriented tangentially to the tubular body, such that a portion of the graft material of the one or more folds is in contact with an outer surface of the tubular body.

For some applications, transitioning the tubular body to the first radially-expanded deployment state includes transitioning the tubular body to the first radially-expanded deployment state such that the graft material is shaped so as to define exactly one or exactly two folds.

For some applications, a greatest internal perimeter of the graft material of a first one of the one or more folds, when the first fold is unfolded when the tubular body is in the second radially-expanded deployment state, is at least 7% of the second greatest internal perimeter. Alternatively or additionally, for some applications, a greatest internal perimeter of the graft material of a second one of the one or more folds, when the second fold is unfolded, is at least 7% of the second greatest internal perimeter.

For some applications, introducing the stent-graft includes introducing the stent-graft into the blood vessel while the graft material is shaped so as to define one or more folds disposed such that at least 50% of the graft material of the folds is radially outside the stent member. For some applications, introducing the stent-graft includes introducing the stent-graft into the blood vessel while the graft material is shaped so as to define one or more folds disposed such that at least 75%, such as 100%, of the graft material of the folds is radially outside the stent member.

For some applications, transitioning the tubular body to the second radially-expanded deployment state includes transitioning the tubular body to the second radially-expanded deployment state such that the graft material of the fluid flow guide is shaped so as to define none of the folds or fewer of the folds than when the tubular body is in the first radially-expanded deployment state.

For some applications, introducing the stent-graft includes introducing the stent-graft into the blood vessel while the one or more folds are oriented tangentially to the tubular body, such that a portion of the graft material of the one or more folds is in contact with an outer surface of the tubular body.

For some applications, introducing the stent-graft includes introducing the stent-graft into the blood vessel while the graft material is shaped so as to define exactly one or exactly two folds.

For some applications, a greatest internal perimeter of the graft material of a first one of the one or more folds, when the first fold is unfolded when the tubular body is in the second radially-expanded deployment state, is at least 7% of the second greatest internal perimeter. Alternatively or additionally, for some applications, a greatest internal perimeter of the graft material of a second one of the one or more folds, when the second fold is unfolded, is at least 7% of the second greatest internal perimeter.

For some applications, transitioning the tubular body to the first radially-expanded deployment state includes transitioning the tubular body to the first radially-expanded deployment state such that the graft material is shaped so as to define one or more folds, and a greatest internal perimeter of the graft material of a first one of the one or more folds, when the first fold is unfolded when the tubular body is in the second radially-expanded deployment state, is at least 7% of the second greatest internal perimeter. For some applications, the first length equals at least 10% of the second greatest internal perimeter.

For some applications, a greatest internal perimeter of the graft material of a second one of the one or more folds, when the second fold is unfolded, is at least 7% of the second greatest internal perimeter.

For some applications, transitioning the tubular body to the second radially-expanded deployment state includes transitioning the tubular body to the second radially-expanded deployment state such that the fluid flow guide is shaped so as to define none of the folds or fewer of the folds than when the tubular body is in the first radially-expanded deployment state.

For some applications, introducing the stent-graft includes introducing the stent-graft into the blood vessel while the graft material is shaped so as to define one or more folds, and a greatest internal perimeter of the graft material of a first one of the one or more folds, when the first fold is unfolded when the tubular body is in the second radially-expanded deployment state, is at least 7% of the second greatest internal perimeter. For some applications, the first length equals at least 10% of the second greatest internal perimeter. For some applications, a greatest internal perimeter of the graft material of a second one of the one or more folds, when the second fold is unfolded, is at least 7% of the second greatest internal perimeter.

For some applications, transitioning the tubular body to the second radially-expanded deployment state includes transitioning the tubular body to the second radially-expanded deployment state such that the fluid flow guide is shaped so as to define none of the folds or fewer of the folds than when the tubular body is in the first radially-expanded deployment state.

For some applications, transitioning the tubular body to the first radially-expanded deployment state includes transitioning the tubular body to the first radially-expanded deployment state such that the graft material is shaped so as to define exactly one or exactly two folds. For some applications, transitioning the tubular body to the first radially-expanded deployment state includes transitioning the tubular body to the first radially-expanded deployment state such the graft material is shaped so as to define exactly one fold.

For some applications, introducing the stent-graft includes introducing the stent-graft into the blood vessel while the graft material is shaped so as to define exactly one or exactly two folds. For some applications, introducing the stent-graft includes introducing the stent-graft into the blood vessel while the graft material is shaped so as to define exactly one fold.

For some applications, providing the endovascular stent-graft includes providing the endovascular stent-graft in which the tubular body further includes a stent member, which includes a plurality of self-expandable flexible structural stent elements, and at least one circumferential expansion element; and transitioning the tubular body to a second radially-expanded deployment state includes causing an increase in a circumferential length of the circumferential expansion element, by applying a force to the stent member, which force is insufficient to cause plastic deformation of the self-expandable flexible structural stent elements and is sufficient to cause plastic deformation of the circumferential expansion element. For some applications, providing the endovascular stent-graft includes providing the endovascular stent-graft in which the circumferential expansion element circumscribes an angle of at least 3 degrees, e.g., at least 5 degrees, when the tubular body is in the first radially-expanded deployment state.

For some applications, providing the endovascular stent-graft includes providing the endovascular stent-graft in which the circumferential expansion element is coupled to at least two of the self-expandable flexible structural stent elements of the expanding portion of the tubular body. For some applications, providing the endovascular stent-graft includes providing the endovascular stent-graft in which a pair of the at least two of the self-expandable flexible structural stent elements to which the circumferential expansion element is coupled are coupled at a peak.

For some applications, providing the endovascular stent-graft includes providing the endovascular stent-graft in which the self-expandable flexible structural stent elements of the stent member are shaped so to define at least one circumferential band at the expanding portion, which band is shaped so as to define a plurality of peaks directed in a first longitudinal direction, alternating with a plurality of troughs directed in a second longitudinal direction opposite the first longitudinal direction. For some applications, providing the endovascular stent-graft includes providing the endovascular stent-graft in which the at least one circumferential expansion element is positioned alongside one of the self-expandable flexible structural stent elements near an element selected from the group consisting of: one of the peaks and one of the troughs. For some applications, providing the endovascular stent-graft includes providing the endovascular stent-graft in which the at least one circumferential expansion element is shaped similarly to a portion of the self-expandable flexible structural stent elements alongside which the at least one circumferential expansion element is positioned.

For some applications, providing the endovascular stent-graft includes providing the endovascular stent-graft in which at least a portion of the fluid flow guide in a vicinity of the circumferential expansion element includes a stretchable fabric, so as to accommodate the increasing of the greatest internal perimeter of the expanding portion.

For some applications, providing the endovascular stent-graft includes providing the endovascular stent-graft in which the circumferential expansion element has a shape selected from the group of shapes consisting of: a U-shape, a V-shape, a W-shape, and an undulating shape, at least when the tubular body is in the first radially-expanded deployment state.

For some applications, transitioning the tubular body to the second radially-expanded deployment state in the blood vessel includes transvascularly introducing a balloon into the tubular body, and inflating the balloon to apply the force from within the tubular body.

For some applications:
the method further includes, after the minimally-invasive secondary intervention procedure, during a minimally-invasive tertiary intervention procedure separate from the primary and the secondary intervention procedures, transitioning the tubular body to a third radially-expanded deployment state in the blood vessel, in which state the expanding portion has a third greatest internal perimeter and forms a blood-tight seal with the wall of the blood vessel, which third greatest internal perimeter is greater than the second greatest internal perimeter,
the balloon is a first one of a plurality of balloons, and
transitioning the tubular body to a third radially-expanded deployment state includes transvascularly introducing a second one of the plurality of balloons into the tubular body, which second balloon has a larger volume than that of the first balloon, and inflating the second balloon to apply the force from within the tubular body.

For some applications, providing the endovascular stent-graft includes providing the endovascular stent-graft in which the circumferential expansion element includes non-elastic stainless steel.

For some applications, providing the endovascular stent-graft includes providing the endovascular stent-graft in which the circumferential expansion element is generally non-elastic. For some applications, providing the endovascular stent-graft includes providing the endovascular stent-graft in which an angular segment of the expanding portion that includes the circumferential expansion element expands and contracts at least 50% less per unit circumferential arc angle than an angular segment of the expanding portion that does not include the circumferential expansion element, as the body cycles between being internally pressurized by (a) fluid having a pressure of 80 mmHg and (b) fluid having a pressure of 120 mmHg

For some applications, providing the endovascular stent-graft includes providing the endovascular stent-graft in which the circumferential expansion element includes a cobalt-chromium alloy.

For some applications:
providing the endovascular stent-graft includes providing the endovascular stent-graft in which the tubular body further includes a stent member, which includes (a) a plurality of self-expandable flexible structural stent elements, which, when unconstrained, are configured to cause the tubular body to assume the second radially-expanded deployment state, and (b) a circumferential expansion prevention element, which is coupled to at least two of the self-expandable flexible structural stent elements of the expanding portion of the tubular body, wherein, when intact, the circumferential expansion prevention element restrains the tubular body in the first radially-expanded deployment state, in which the expanding portion has a first greatest internal perimeter, and
transitioning the tubular body to a second radially-expanded deployment state includes detaching or severing the circumferential expansion prevention element, so that it does not restrain the tubular body in the first radially-expanded deployment state.

For some applications, providing the endovascular stent-graft includes providing the endovascular stent-graft in which the circumferential expansion prevention element includes an element selected from the group consisting of: a suture, a wire, a hook, a loop, and a helix.

For some applications, providing the endovascular stent-graft includes providing the endovascular stent-graft in which the circumferential expansion prevention element circumscribes an angle of at least 3 degrees, e.g., at least 5 degrees, when the tubular body is in the first radially-expanded deployment state.

For some applications, providing the endovascular stent-graft includes providing the endovascular stent-graft in which the self-expandable flexible structural stent elements of the stent member are shaped so to define at least one circumferential band at the expanding portion, which band is shaped so as to define a plurality of peaks directed in a first longitudinal direction, alternating with a plurality of troughs directed in a second longitudinal direction opposite the first longitudinal direction, and the circumferential expansion prevention element is coupled to the at least two of the structural elements within 30% of a diameter of the body in its first radially-expanded state of respective ones of the peaks. For some applications, providing the endovascular stent-graft includes providing the endovascular stent-graft in which the circumferential expansion prevention element is coupled to the at least two of the structural elements at respective ones of the peaks.

The present invention will be more fully understood from the following detailed description of applications thereof, taken together with the drawings, in which:

### BRIEF DESCRIPTION OF THE DRAWINGS

FIGS. 1A-B are schematic illustrations of an endovascular stent-graft, in accordance with an application of the present invention;

FIGS. 2A-B are schematic illustrations of another configuration of the endovascular stent-graft of FIGS. 1A-B, in accordance with an application of the present invention;

FIGS. 3A-B are schematic illustrations of another endovascular stent-graft, in accordance with an application of the present invention;

FIGS. 4A-B are schematic illustrations of an endovascular stent-graft system, in accordance with an application of the present invention;

FIGS. 5A-B are schematic illustrations of another endovascular stent-graft system, in accordance with an application of the present invention;

FIGS. 6A-B are schematic illustrations of yet another endovascular stent-graft system, in accordance with an application of the present invention;

FIGS. 7A-B are schematic illustrations of an exemplary method for deploying the stent-graft of FIGS. 1A-B and 2A-B, in accordance with an application of the present invention; and

FIGS. 8A-B are schematic illustrations of an exemplary method for deploying the stent-graft of FIGS. 4A-B, in accordance with an application of the present invention.

### DETAILED DESCRIPTION OF APPLICATIONS

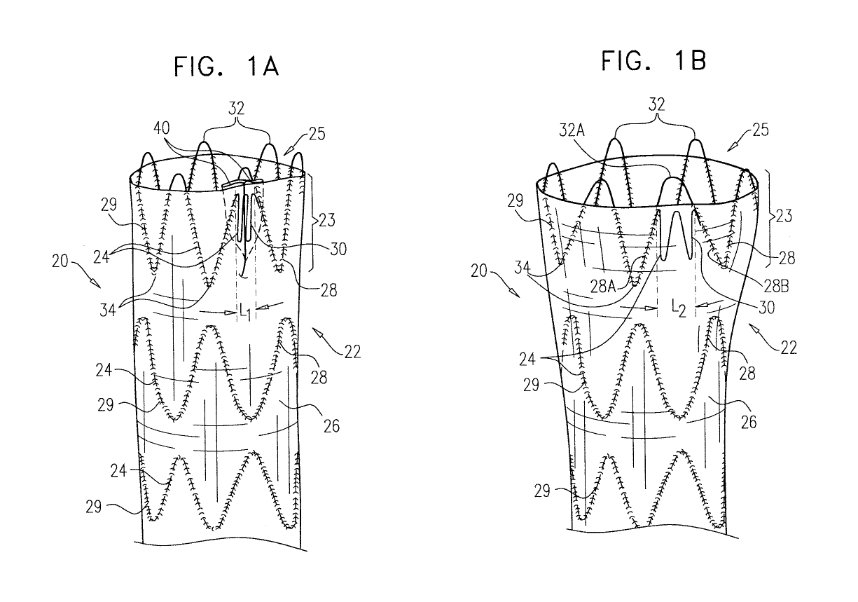

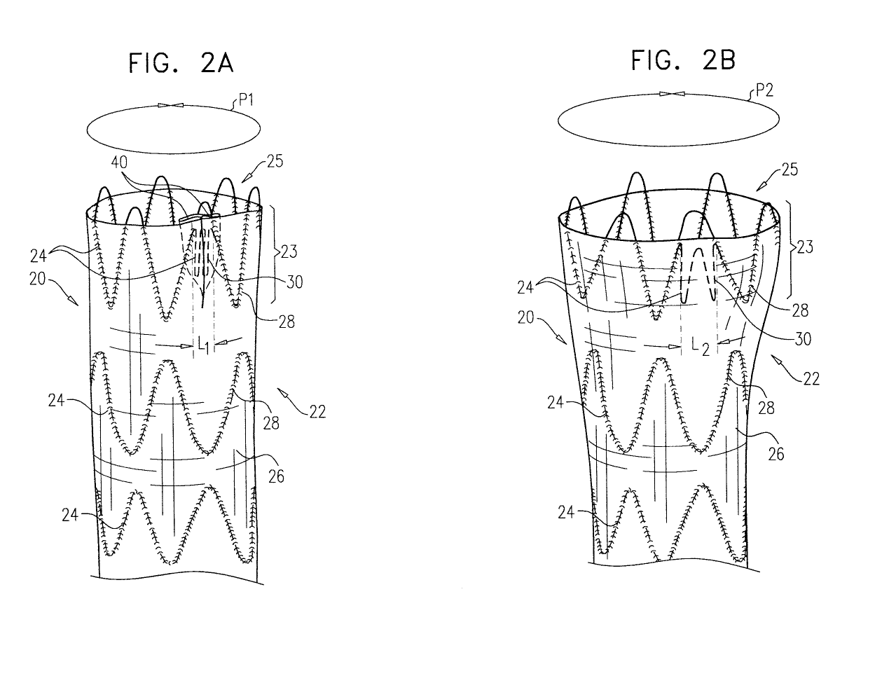

FIGS. 1A-B and 2A-B are schematic illustrations of an endovascular stent-graft 20, in accordance with an application of the present invention. Stent-graft 20 comprises a generally tubular body 22. Body 22 is configured to assume (a) a radially-compressed delivery state, typically when the body is initially positioned in a delivery catheter, and (b) at least first and second radially-expanded deployment states. Body 22 typically assumes the first radially-expanded deployment state upon deployment from the delivery catheter, and the second radially-expanded delivery state after deployment, typically during a minimally-invasive secondary intervention procedure. FIGS. 1A and 2A show the stent-graft with body 22 in its first radially-expanded deployment state, and FIGS. 1B and 2B show the stent-graft with body 22 in its second radially-expanded deployment state.

Body 22 is shaped so as to define a stepwise expanding portion 23, a greatest internal perimeter of which increases as body 22 transitions from the first radially-expanded delivery state to the second radially-expanded delivery state. (The “greatest” internal perimeter of the expanding portion means the internal perimeter as measured at the longitudinal location along the expanding portion that has the greatest internal perimeter.) For some applications, expanding portion 23 is disposed at a longitudinal end 25 of body 22, as shown in FIGS. 1A-B and 2A-B. For example, all of expanding portion 23 may be disposed with a distance of longitudinal end 25, measured along an axis of body 22, which distance is less than 30%, such as less than 25%, of an axial length of body 22. Alternatively or additionally, the distance is less than 120%, such as less than 80%, of an average diameter of the expanding portion when body 22 is in the first radially-expanded state. For other applications, the expanding portion is disposed elsewhere along stent-graft 20.

Body 22 comprises a stent member 24, and, typically, a generally tubular fluid flow guide 26. The fluid flow guide and the stent member are attached to each other, such as by suturing or stitching. The fluid flow guide is configured to accommodate the increase in the greatest internal perimeter of expanding portion 23, as described hereinbelow. The stent member may be attached to an internal and/or an external surface of the fluid flow guide.

Stent member 24 comprises a plurality of self-expandable flexible structural stent elements 28, which are either indirectly connected to one another by the fluid flow guide (as shown), and/or interconnected with one another (configuration not shown). Optionally, a portion of structural stent elements 28 may be attached (e.g., sutured) to the internal surface of the fluid flow guide, and another portion to the external surface of the fluid flow guide. Structural stent elements 28 comprise a self-expanding material, such as a self-expanding metal, such that body 22 is self-expandable. Typically, structural stent elements 28 comprise one or more metallic alloys, such as one or more superelastic metal alloys, a shape memory metallic alloy, and/or Nitinol. Typically, stent-graft 20 is configured to self-expand from the delivery state to the first radially-expanded deployment state. For example, stent member 24 may be heat-set to cause stent-graft 20 to self-expand from the delivery state to the first radially-expanded deployment state.

For some applications, flexible structural stent elements 28 of stent member 24 are shaped so to define at least one circumferential band 29 at expanding portion 23, such as exactly one circumferential band 29 or a plurality of circumferential bands 29. Circumferential band 29 is shaped so as to define a plurality of peaks 32 directed in a first longitudinal direction, alternating with a plurality of troughs 34 directed in a second longitudinal direction opposite the first longitudinal direction. Circumferential band 29 may be serpentine-shaped. Typically, stent member 24 is shaped so as to further define one or more additional circumferential bands 29 at respective longitudinal locations other than expanding portion 23, as shown in FIGS. 1A-2B.

Stent member 24 further comprises at least one circumferential expansion element 30, which is coupled to at least two of self-expandable flexible structural stent elements 28 of expanding portion 23 of tubular body 22. For some applications, circumferential expansion element 30 has a shape selected from the group of shapes consisting of: a U-shape, a V-shape, a W-shape, and an undulating shape, at least when tubular body 22 is in the first radially-expanded deployment state. For some applications, as labeled in FIG. 1B, a pair of the at least two of self-expandable flexible structural stent elements 28A and 28B to which circumferential expansion element 30 is coupled are coupled at a peak 32A. Circumferential expansion element 30 may be disposed either radially outside fluid flow guide 26, as shown in FIGS. 1A-B, or radially inside fluid flow guide 26, as shown in FIGS. 2A-B. Typically, circumferential expansion element 30 is attached to the fluid flow guide, e.g., sutured to the fluid flow guide (such as in applications in which the fluid flow guide comprises polyester), or encapsulated within the fluid flow guide (such as in applications in which the fluid flow guide comprises ePTFE).

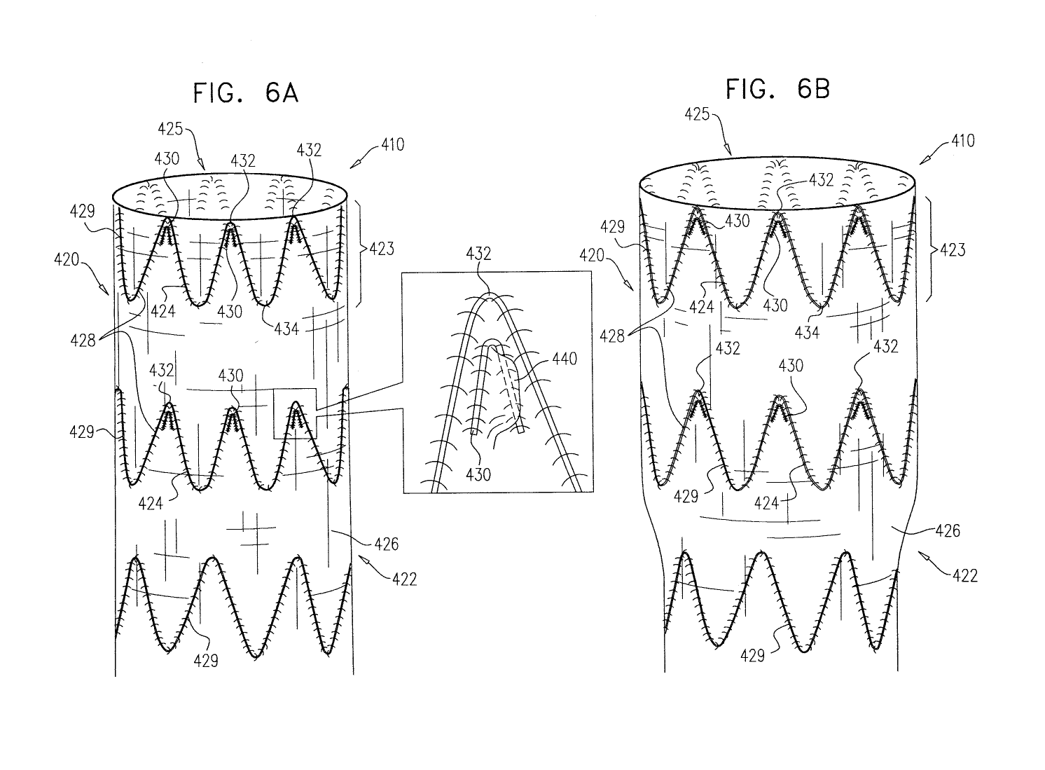

For some applications, stent member 24 comprises a plurality of circumferential expansion elements 30. For some applications, circumferential expansion elements 30 are alternatively or additionally coupled to at least two of self-expandable flexible structural stent elements 28 of one or more circumferential bands 29 positioned at respective longitudinal locations other than expanding portion 23, such as described hereinbelow with reference to FIGS. 6A-B regarding circumferential expansion elements 430. Alternatively or additionally, for some applications, circumferential expansion elements 30 are coupled to a plurality of circumferential bands 29, respectively.

For some applications, circumferential expansion element 30 is generally non-elastic. Alternatively or additionally, circumferential expansion element 30 is substantially less elastic than structural stent elements 28. For example, an angular segment of expanding portion 23 that comprises circumferential expansion element 30 may expand and contract at least 30% less, such as at least 50% less, e.g., at least 67% less, per unit circumferential arc angle than an angular segment of expanding portion 23 that does not comprise circumferential expansion element 30, as body 22 cycles between being internally pressurized by (a) fluid having a pressure of 80 mmHg, typically by blood during diastole in an adult human, and (b) fluid having a pressure of 120 mmHg, typically by blood during systole in an adult human. For example, circumferential expansion element 30 may comprise non-elastic stainless steel, or a cobalt-chromium alloy.

Fluid flow guide 26 comprises a graft material, i.e., at least one biologically-compatible substantially blood-impervious flexible sheet. The flexible sheet may comprise, for example, a polyester, a polyethylene (e.g., a poly-ethylene-terephthalate), a polymeric film material (such as a fluoropolymer, e.g., polytetrafluoroethylene), a polymeric textile material (e.g., woven polyethylene terephthalate (PET)), natural tissue graft (e.g., saphenous vein or collagen), Polytetrafluoroethylene (PTFE), ePTFE, Dacron, or a combination of two or more of these materials. The graft material optionally is woven. For some applications, the graft material of fluid flow guide 26 is generally non- or minimally-elastic.

Stent member 24 is configured such that application of a force thereto, which is insufficient to cause plastic deformation of self-expandable flexible structural stent elements 28 and is sufficient to cause plastic deformation of circumferential expansion element 30, causes plastic deformation of and an increase in a circumferential length L of circumferential expansion element 30, from a first length L1, as shown in FIGS. 1A and 2A, to a second length L2, as shown in FIGS. 1B and 2B. This increase in length transitions tubular body 22 from the first radially-expanded deployment state, as shown in FIGS. 1A and 2A, to the second radially-expanded deployment state, as shown in FIGS. 1B and 2B, thereby increasing a greatest internal perimeter of expanding portion 23, from a first greatest internal perimeter P1 (labeled in FIG. 2A) to a second greatest internal perimeter P2 (labeled in FIG. 2B). Because of the plastic deformation, circumferential expansion element 30 retains its increased length L2 even after the force is no longer applied.

Typically, circumferential expansion element 30, or, for applications in which stent member 24 comprises a plurality of circumferential expansion elements 30, circumferential expansion elements 30 collectively circumscribe an aggregate angle of at least 20 degrees, when tubular body 22 is in the first radially-expanded deployment state, as shown in FIGS. 1A and 2A. For example, the angle may be at least 40 degrees, such as at least 90 degrees. Typically, each of circumferential expansion elements 30 circumscribes an angle of at least 3 degrees, such as at least 5 degrees, when tubular body 22 is in the first radially-expanded deployment state, as shown in FIGS. 1A and 2A. For some applications, when tubular body 22 is the second radially-expanded deployment state, circumferential expansion element 30 circumscribes an angle that is capable of attaining a value that is at least 30% greater than when tubular body 22 is the first radially-expanded deployment state. For some applications, a resistance of fluid flow guide 26 to lateral expansion is less than 70%, e.g., less than 30%, of a resistance of circumferential expansion element 30 to circumferential expansion.

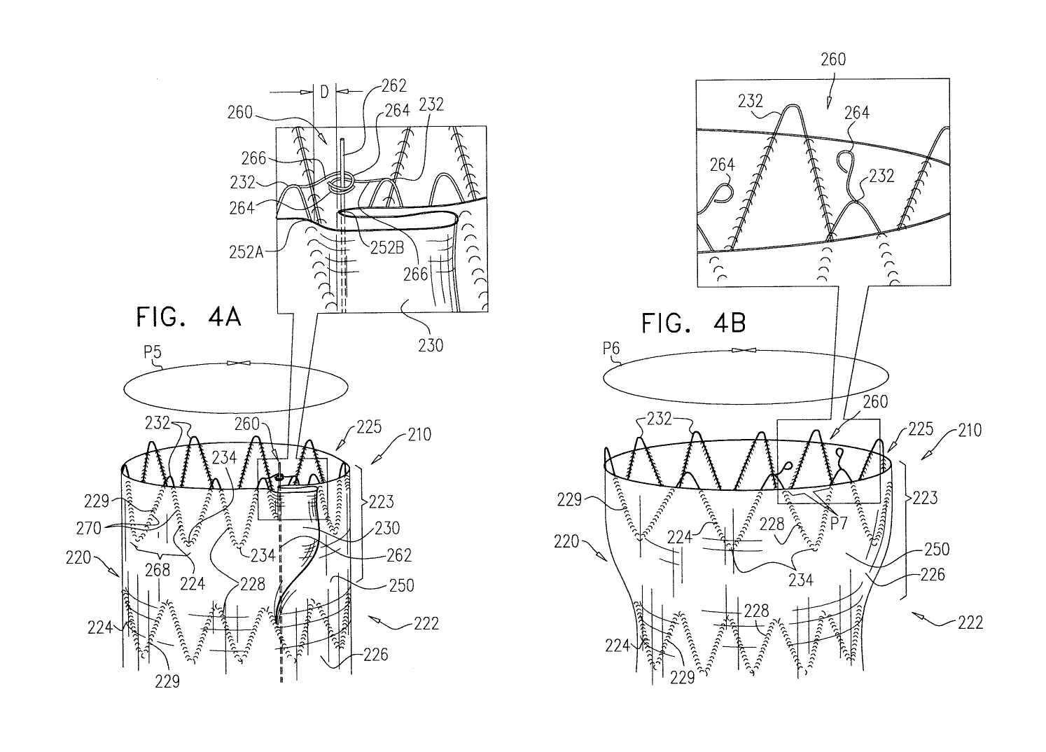

As mentioned above, fluid flow guide 26 is configured to accommodate the increase in the greatest internal perimeter of expanding portion 23. For some applications, in order to provide such accommodation, when tubular body 22 is in the first radially-expanded deployment state, fluid flow guide 26 is shaped so as to define one or more folds 40 in a vicinity of circumferential expansion element 30, such as shown in FIGS. 1A and 2A. For some applications, such as shown in FIGS. 1A and 2A, when tubular body 22 is in the first radially-expanded deployment state, the one or more folds are disposed radially inside stent member 24. For other applications, similar to the configurations shown in FIGS. 4A, 5A, and 8A, when tubular body 22 is in the first radially-expanded deployment state, the one or more folds are disposed radially outside stent member 24.

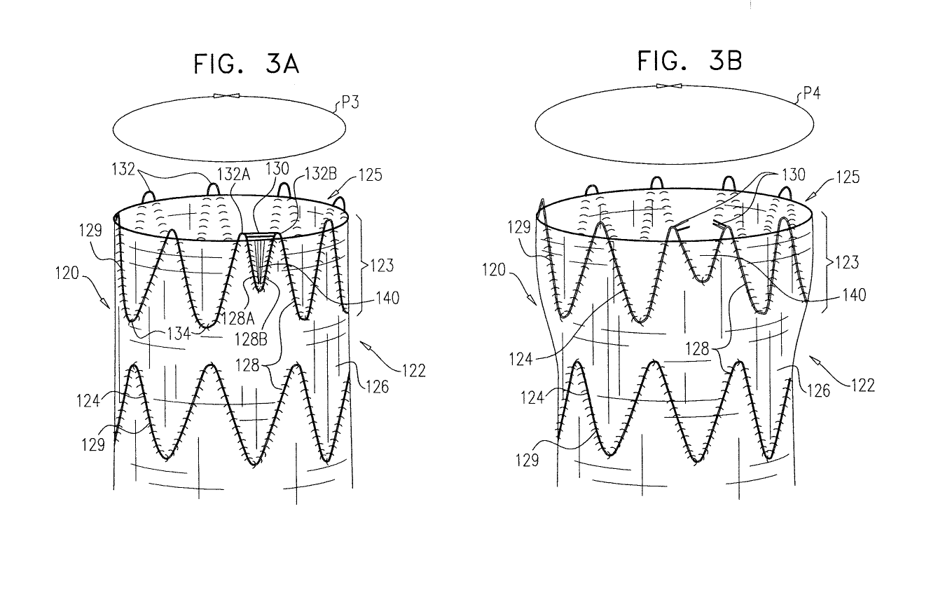

Alternatively, for some applications, in order to provide such accommodation, at least a portion of fluid flow guide 26 in a vicinity of circumferential expansion element 30 comprises a stretchable fabric (this configuration is not shown in FIGS. 1A-B and 2A-B, but is similar to the configuration shown in FIG. 3A, described hereinbelow). For example, the stretchable fabric may comprise expanded polytetrafluoroethylene (ETFE).

For some applications, fluid flow guide 26, other than the portion in the vicinity of circumferential expansion element 30, comprises a fabric that is less elastic than the stretchable fabric. For example, the fabric of an angular segment of expanding portion 23 that comprises circumferential expansion element 30 may expand and contract at least 30% less, such as at least 50% less, e.g., at least 67% less, per unit circumferential arc angle than the fabric of an angular segment of expanding portion 23 that does not comprise circumferential expansion element 30, as body 22 cycles between being internally pressurized by (a) fluid having a pressure of 80 mmHg, typically by blood during diastole in an adult human, and (b) fluid having a pressure of 120 mmHg, typically by blood during systole in an adult human.

Reference is now made to FIGS. 3A-B, which are schematic illustrations of an endovascular stent-graft 120, in accordance with an application of the present invention. Stent-graft 120 comprises a generally tubular body 122. Body 122 is configured to assume (a) a radially-compressed delivery state, typically when the body is initially positioned in a delivery catheter, and (b) at least first and second radially-expanded deployment states. Body 122 typically assumes the first radially-expanded deployment state upon deployment from the delivery catheter, and the second radially-expanded delivery state after deployment, typically during a minimally-invasive secondary intervention procedure. FIG. 3A shows the stent-graft with body 122 in its first radially-expanded deployment state, and FIG. 3B shows the stent-graft with body 122 in its second radially-expanded deployment state.

Body 122 is shaped so as to define a stepwise expanding portion 123, a greatest internal perimeter of which increases as body 122 transitions from the first radially-expanded delivery state to the second radially-expanded delivery state. (The “greatest” internal perimeter of the expanding portion means the internal perimeter as measured at the longitudinal location along the expanding portion that has the greatest internal perimeter.) For some applications, expanding portion 123 is disposed at a longitudinal end 125 of body 122, as shown in FIGS. 3A-B. For example, all of expanding portion 123 may be disposed with a distance of longitudinal end 125, measured along an axis of body 122, which distance is less than 30%, such as less than 25%, of an axial length of body 122. Alternatively or additionally, the distance is less than 120%, such as less than 80%, of an average diameter of the expanding portion when body 122 is in the first radially-expanded state. For other applications, the expanding portion is disposed elsewhere along stent-graft 120.

Body 122 comprises a stent member 124, and, typically, a generally tubular fluid flow guide 126. The fluid flow guide and the stent member are attached to each other, such as by suturing or stitching. The fluid flow guide is configured to accommodate the increase in the greatest internal perimeter of expanding portion 123, as described hereinbelow. The stent member may be attached to an internal and/or an external surface of the fluid flow guide.

Stent member 124 comprises a plurality of self-expandable flexible structural stent elements 128, which are either indirectly connected to one another by the fluid flow guide (as shown), and/or interconnected with one another (configuration not shown).

Optionally, a portion of structural stent elements 128 may be attached (e.g., sutured) to the internal surface of the fluid flow guide, and another portion to the external surface of the fluid flow guide. For some applications, self-expandable flexible structural stent elements 128 of stent member 124 are shaped so to define at least one circumferential band 129 at expanding portion 123, such as exactly one circumferential band 129 or a plurality of circumferential bands 129. Circumferential band 129 is shaped so as to define a plurality of peaks 132 directed in a first longitudinal direction, alternating with a plurality of troughs 134 directed in a second longitudinal direction opposite the first longitudinal direction. Circumferential band 129 may be serpentine-shaped. Typically, stent member 124 is shaped so as to further define one or more additional circumferential bands 129 at respective longitudinal locations other than expanding portion 123, as shown in FIGS. 3A-B.

Self-expandable flexible structural stent elements 128 of stent member 124, when unconstrained, are configured to cause tubular body 122 to assume the second radially-expanded deployment state. Structural stent elements 128 comprise a self-expanding material, such as a self-expanding metal. Typically, structural stent elements 128 comprise one or more metallic alloys, such as one or more superelastic metal alloys, a shape memory metallic alloy, and/or Nitinol. Typically, stent-graft 120 is configured to self-expand from the delivery state to the first radially-expanded deployment state. For example, stent member 124 may be heat-set to cause stent-graft 120 to self-expand from the delivery state to the first radially-expanded deployment state.

Stent-graft 120 further comprises at least one circumferential expansion prevention element 130, which is coupled to at least two of self-expandable flexible structural stent elements 128A and 128B of expanding portion 123 of tubular body 122. When intact, circumferential expansion prevention element 130 restrains tubular body 122 in the first radially-expanded deployment state, in which expanding portion 123 has a first greatest internal perimeter P3. When detached and/or severed, such as by application of a force that increases a distance between stent elements 128A and 128B, circumferential expansion prevention element 130 does not restrain tubular body 122 in the first radially-expanded deployment state, such that the tubular body transitions from the first radially-expanded deployment state to the second radially-expanded deployment state. In the second radially-expanded deployment state, expanding portion 123 has a second greatest internal perimeter P4, which is greater than first greatest internal perimeter P3.

For some applications, stent-graft 120 comprises a plurality of circumferential expansion prevention elements 130. For some applications, circumferential expansion prevention elements 130 are alternatively or additionally coupled to at least two of self-expandable flexible structural stents elements 128 of one or more circumferential bands 129 positioned at respective longitudinal locations other than expanding portion 123, such as described hereinbelow with reference to FIGS. 6A-B regarding circumferential expansion elements 430.

For some applications, circumferential expansion prevention element 130 comprises a suture, a wire (e.g., comprising stainless steel, nitinol, poly propylene, polyester, ePTFE), a hook, a loop, or a helix. Circumferential expansion prevention element 130 is detached and/or severed, such as by cutting or breaking thereof, either at a location along circumferential expansion prevention element 130, and/or at the interface with one or both of self-expandable flexible structural stent elements 128A and 128B. For example, a cutting tool may be used, or a balloon may be used to apply a force sufficient to detach and/or sever the element, by increasing a distance between stent elements 128A and 128B to which element 130 is coupled.

For some applications, circumferential expansion prevention element 130 is coupled to the at least two of the structural elements within a distance of respective ones of the peaks 132A and 132B, which distance equals 30% of a diameter of body 22 in its first radially-expanded state. For example, the distance may equal zero, i.e., circumferential expansion prevention element 130 may be coupled to at least two peaks 132A and 132B of two structural stent elements 128A and 128B, as shown in FIG. 3B. For other applications, circumferential expansion prevention element 130 is coupled to two structural stent elements 128A and 128B at respective sites thereof other than peaks 132A and 132B (configuration not shown). Circumferential expansion prevention element 130 may be disposed either radially outside or radially inside fluid flow guide 126.

Fluid flow guide 126 comprises a graft material, i.e., at least one biologically-compatible substantially blood-impervious flexible sheet. The flexible sheet may comprise, for example, a polyester, a polyethylene (e.g., a poly-ethylene-terephthalate), a polymeric film material (such as a fluoropolymer, e.g., polytetrafluoroethylene), a polymeric textile material (e.g., woven polyethylene terephthalate (PET)), natural tissue graft (e.g., saphenous vein or collagen), Polytetrafluoroethylene (PTFE), ePTFE, Dacron, or a combination of two or more of these materials. The graft material optionally is woven. For some applications, the graft material of fluid flow guide 126 is generally non- or minimally-elastic.

Typically, circumferential expansion prevention element 130, or, for applications in which stent-graft 120 comprises a plurality of circumferential expansion prevention elements 130, circumferential expansion prevention elements 130 collectively circumscribe an aggregate angle of at least 40 degrees, when tubular body 122 is in the first radially-expanded deployment state, as shown in FIG. 3A. For example, the angle may be at least 50 degrees, such as at least 90 degrees. Typically, each of circumferential expansion prevention elements 130 circumscribes an angle of at least 3 degrees, such as at least 5 degrees, when tubular body 122 is in the first radially-expanded deployment state, as shown in FIG. 3A.

As mentioned above, fluid flow guide 126 is configured to accommodate the increase in the greatest internal perimeter of expanding portion 123. For some applications, in order to provide such accommodation, at least a portion 140 of fluid flow guide 126 in a vicinity of circumferential expansion prevention element 130 comprises a stretchable fabric For example, the stretchable fabric may comprise expanded polytetrafluoroethylene (ETFE). For some applications, fluid flow guide 126, other than the portion in the vicinity of circumferential expansion prevention element 130, comprises a fabric that is less elastic than the stretchable fabric. For example, the fabric of an angular segment of expanding portion 123 that comprises circumferential expansion prevention element 130 may expand and contract at least 30% less, such as at least 50% less, e.g., at least 67% less, per unit circumferential arc angle than the fabric of an angular segment of expanding portion 123 that does not comprise circumferential expansion prevention element 130, as body 122 cycles between being internally pressurized by (a) fluid having a pressure of 80 mmHg, typically by blood during diastole in an adult human, and (b) fluid having a pressure of 120 mmHg, typically by blood during systole in an adult human.

Alternatively, for some applications, in order to provide such accommodation, when tubular body 122 is in the first radially-expanded deployment state, fluid flow guide 126 is shaped so as to define one or more folds in a vicinity of circumferential expansion prevention element 130 (this configuration is not shown in FIGS. 3A, but is similar to the configuration shown in FIGS. 1A and 2A). For some applications, when the tubular body is in the first radially-expanded deployment state, the one or more folds are disposed radially outside stent member 124, while for some applications, when the tubular body is in the first radially-expanded deployment state, the one or more folds are disposed radially inside stent member 124.

Reference is now made to FIGS. 4A-B, which are schematic illustrations of an endovascular stent-graft system 210, in accordance with an application of the present invention. Stent-graft system 210 comprises a stent-graft 220, which comprises a generally tubular body 222. Body 222 is configured to assume (a) a radially-compressed delivery state, typically when the body is initially positioned in a delivery catheter, and (b) at least first and second radially-expanded deployment states. Body 222 typically assumes the first radially-expanded deployment state upon deployment from the delivery catheter, and the second radially-expanded delivery state after deployment, typically during a minimally-invasive secondary intervention procedure. FIG. 4A shows the stent-graft with body 222 in its first radially-expanded deployment state, and FIG. 4B shows the stent-graft with body 222 in its second radially-expanded deployment state.

Body 222 is shaped so as to define a stepwise expanding portion 223, a greatest internal perimeter of which increases as body 222 transitions from the first radially-expanded delivery state to the second radially-expanded delivery state. Tubular body 22, when in the first radially-expanded deployment state, is restrained from transitioning to the second radially-expanded deployment state. (The “greatest” internal perimeter of the expanding portion means the internal perimeter as measured at the longitudinal location along the expanding portion that has the greatest internal perimeter.) For some applications, expanding portion 223 is disposed at a longitudinal end 225 of body 222, as shown in FIGS. 4A-B. For example, all of expanding portion 223 may be disposed with a distance of longitudinal end 225, measured along an axis of body 222, which distance is less than 30%, such as less than 25%, of an axial length of body 222. Alternatively or additionally, the distance is less than 120%, such as less than 80%, of an average diameter of the expanding portion when body 222 is in the first radially-expanded state. For other applications, the expanding portion is disposed elsewhere along stent-graft 220.

Body 222 comprises a self-expandable flexible stent member 224, and a generally tubular fluid flow guide 226. The fluid flow guide is attached to stent member 224, such as by suturing or stitching. The fluid flow guide is configured to accommodate the increase in the greatest internal perimeter of expanding portion 223, as described hereinbelow. The stent member may be attached to an internal and/or an external surface of the fluid flow guide.

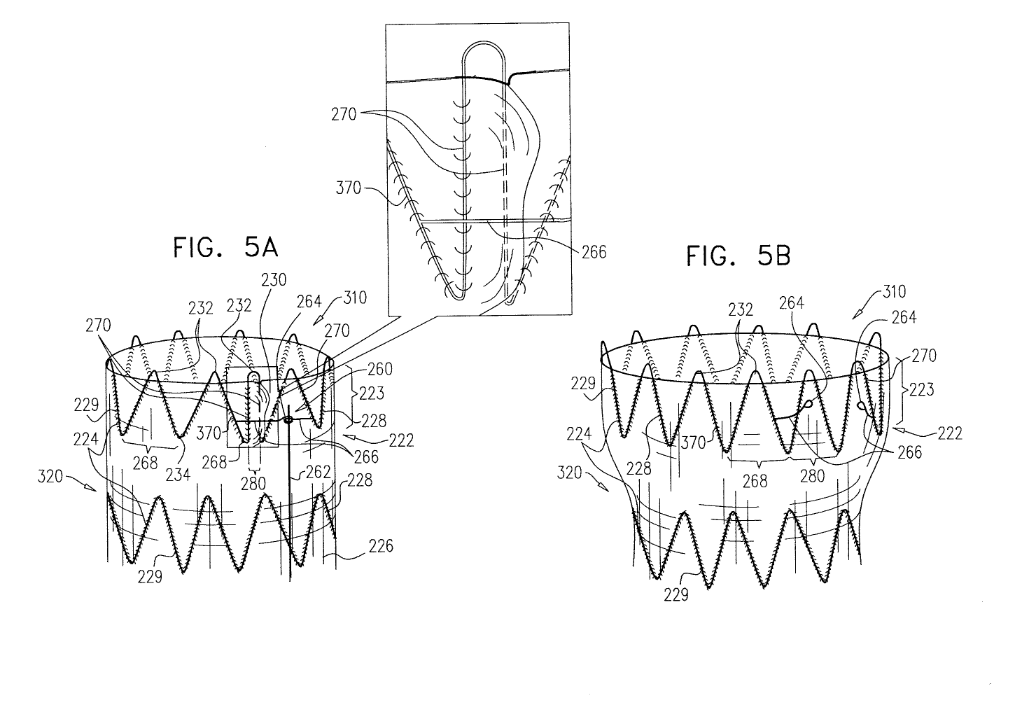

Stent member 224 comprises a plurality of self-expandable flexible structural stent elements 228, which are either indirectly connected to one another by the fluid flow guide (as shown), and/or interconnected with one another (configuration not shown). Optionally, a portion of structural stent elements 228 may be attached (e.g., sutured) to the internal surface of the fluid flow guide, and another portion to the external surface of the fluid flow guide. For some applications, self-expandable flexible structural stent elements 228 of stent member 224 are shaped so to define at least one circumferential band 229 at expanding portion 223, such as exactly one circumferential band 229 or a plurality of circumferential bands 229. Circumferential band 229 is shaped so as to define a plurality of peaks 232 directed in a first longitudinal direction, alternating with a plurality of troughs 234 directed in a second longitudinal direction opposite the first longitudinal direction. Circumferential band 229 may be serpentine-shaped. Typically, stent member 224 is shaped so as to further define one or more additional circumferential bands 229 at respective longitudinal locations other than expanding portion 223, as shown in FIGS. 4A-B (and FIGS. 5A-B, described hereinbelow).

Self-expandable flexible structural stent elements 228 of stent member 224, when unconstrained, are configured to cause tubular body 222 to assume the second radially-expanded deployment state. Structural stent elements 228 comprise a self-expanding material, such as a self-expanding metal, such that body 222 is self-expandable. Typically, structural stent elements 228 comprise one or more metallic alloys, such as one or more superelastic metal alloys, a shape memory metallic alloy, and/or Nitinol. Typically, stent-graft 220 is configured to self-expand from the delivery state to the first radially-expanded deployment state. For example, stent member 224 may be heat-set to cause stent-graft 220 to self-expand from the delivery state to the first radially-expanded deployment state.

Fluid flow guide 226 comprises a graft material 250, i.e., at least one biologically-compatible substantially blood-impervious flexible sheet. The flexible sheet may comprise, for example, a polyester, a polyethylene (e.g., a poly-ethylene-terephthalate), a polymeric film material (such as a fluoropolymer, e.g., polytetrafluoroethylene), a polymeric textile material (e.g., woven polyethylene terephthalate (PET)), natural tissue graft (e.g., saphenous vein or collagen), Polytetrafluoroethylene (PTFE), ePTFE, Dacron, or a combination of two or more of these materials. The graft material optionally is woven. For some applications, the graft material of fluid flow guide 226 is generally non- or minimally-elastic.

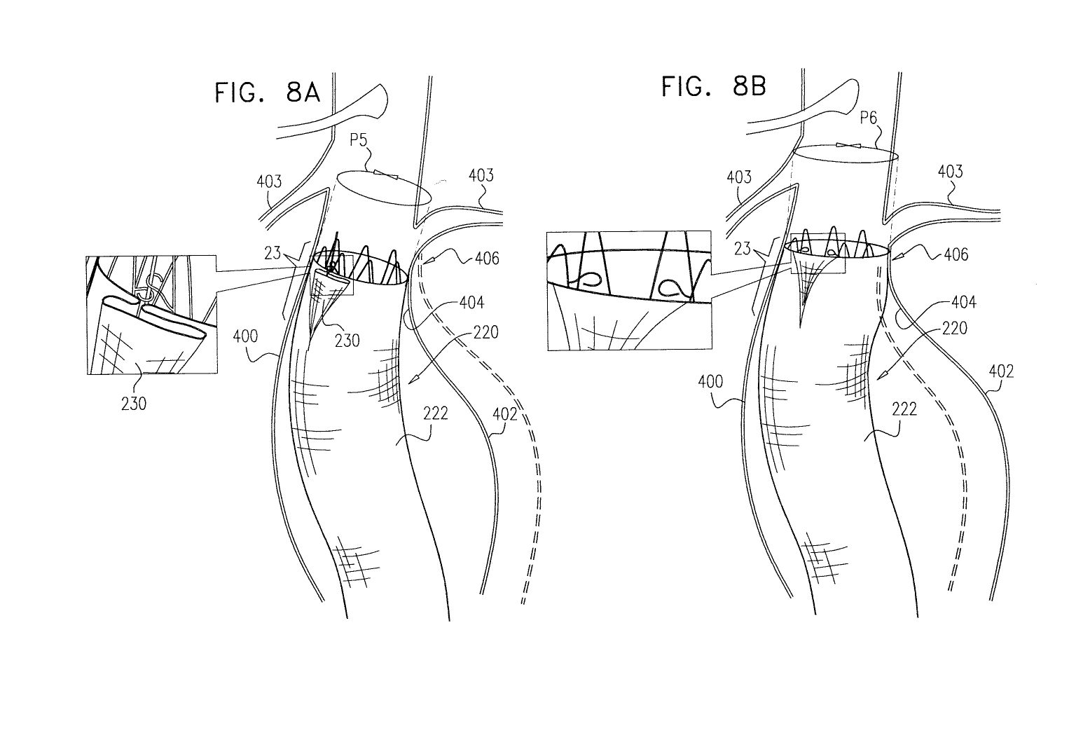

As shown in FIG. 4A, graft material 250 of fluid flow guide 226 is shaped so as to define, when tubular body 222 is in the first radially-expanded deployment state, one or more folds 230. As used in the present application, including in the claims, a “fold” is a portion of graft material 250 that is at least doubled upon itself such that two end portions 252A and 252B of the fold touch or are near each other at the surface generally defined by tubular body 222. (The phrase “at least” doubled is to be understood as including multiple doubling of the graft material upon itself, so long as only the two end portions 252A and 252B of the fold are positioned at the surface generally defined by tubular body 222. For example, the fold may be shaped like a generally flattened Greek lower-case omega (w) or epsilon (s).) For some applications, when stent-graft 220 is in the first radially-expanded deployment state, a distance between end portions 252A and 252B, measured at longitudinal end 225 of body 222, is less than 50%, such as less than 20%, of a first greatest internal perimeter P5 of expanding portion 223. Each fold may be disposed circumferentially in one direction (clockwise or counterclockwise, such as shown in FIG. 4A, or both clockwise and counterclockwise, such as shown in FIG. 8A, described hereinbelow). Thus, in accordance with this definition of “fold,” each of the tubular bodies shown in FIGS. 4A and 8A, as well as FIGS. 1A, 2A, 5A, and 7A, defines exactly one fold. In contrast, tubular body 422, shown in FIG. 6A, defines a plurality of folds 440, one for each circumferential expansion element 430 (for clarity of illustration, only one of these folds is shown clearly, in the enlargement).

For some applications, as shown in FIG. 4A, when tubular body 222 is in the first radially-expanded deployment state, one or more folds 230 are oriented tangentially to tubular body 222, such that a portion of graft material 250 of the one or more folds is in contact with an outer surface of tubular body 222. For some applications, at least when tubular body 222 is in the radially-compressed delivery state, the one or more folds are removably secured to the outer surface of the tubular body. For example, stent-graft system 210 may further comprise a securing mechanism, which removably secures the folds to the outer surface of the tubular body. Alternatively or additionally, stent-graft system 210 may further comprise a bio-dissolvable adhesive, e.g. cyanoacrylate, which removably secures the folds to the outer surface of the tubular body.

For some applications, one or more folds 230 are disposed such that at least 50%, e.g., at least 75%, such as 100%, of graft material 250 of folds 230 is radially outside stent member 224. Disposing of the folds mostly or entirely outside of the stent member reduces or prevents any interfere by the folds with the flow of blood through fluid flow guide 226. If the folds instead extended mostly or entirely into the lumen of the fluid flow guide, the folds would reduce the effective cross-section of the lumen and potentially interfere with blood flow. Although disposing the folds entirely outside the stent member provides the greatest reduction in potential interference with blood flow, this is not always possible because of design considerations. For some applications, graft material 250 is shaped so as to define exactly one or exactly two folds 230 when tubular body 222 is in the first radially-expanded deployment state. For some applications, expanding portion 223 comprises a plurality of circumferential bands 229, and a plurality of the folds 230 are disposed the plurality of circumferential bands 229, respectively (configuration not shown).

Typically, when tubular body 222 is in the second radially-expanded deployment state, graft material 250 of fluid flow guide 226 is shaped so as to define none of folds 230 (as shown in FIG. 4B) or fewer of folds 230 than when the tubular body is in the first radially-expanded deployment state. The portion(s) of the graft material that define folds 230 when the tubular body is in the first radially-expanded deployment state may remain somewhat protruding from stent member 224 even when the tubular body has transitioned to the second radially-expanded deployment state, but is no longer folded.

As body 222 transitions from the first radially-expanded delivery state to the second radially-expanded delivery state, a greatest internal perimeter of expanding portion 223 increases from first greatest internal perimeter P5 to a second greatest internal perimeter P6. For some applications, second greatest internal perimeter P6 of the expanding portion is at least 10% greater than first greatest internal perimeter P5.

For some applications, each of one or more folds 230 is relatively large with respect to the greatest internal perimeter of the expanding portion, in order to provide a large circumferential buffer for expansion of the expanding portion after implantation.

For example, a greatest internal perimeter P7 of graft material 250 of a first one of one or more folds 230, when the first fold is unfolded when tubular body 222 is in the second radially-expanded deployment state, may be equal to at least 7% of second greatest internal perimeter P6, such as at least 10%, e.g., at least 12%. For some applications in which graft material 250 defines at least two folds 230, a greatest internal perimeter of graft material 250 of a second one of one or more folds 230, when the second fold is unfolded, may be equal to at least 7% of second greatest internal perimeter P6, such as at least 10%, e.g., at least 12%.

Typically, each of one or more folds 230 substantially protrudes from or into the stent-graft, i.e., is not a relatively small concavity, convexity, wrinkle, or any other type of deviation from circularity (or ellipticity, in the broader sense) in the graft material of the stent-graft. For example, when stent-graft 20 is in the first radially-expanded deployment state, as shown in FIG. 4A, a length of a fold 230, measured along the graft material of the fold at longitudinal end 225 of body 222 between end portions 252A and 252B of the fold, may be equal to at least 140%, such as at least 167%, at least 300%, or at least 500%, of a distance D between end portions 252A and 252B of the fold at longitudinal end 225. These relative dimensions may also be provided for the folds of the other configurations described herein.

For some applications, the second fold is unfolded when tubular body 222 is in the second radially-expanded deployment state. For other applications, the second fold remains folded when tubular body 222 is in the second radially-expanded deployment state, and is unfolded when tubular body 222 transitions to a third radially-expanded deployment state in which expanding portion 223 has an even greater greatest internal perimeter than second greatest internal perimeter P6.

For some applications, stent-graft system 210 further comprises a locking mechanism 260, which is configured to assume a locked state which restrains tubular body 222 in the first radially-expanded deployment state, such as shown in FIG. 4A, and a released state, which allows tubular body 222 to transition to the second radially-expanded deployment state, such as shown in FIG. 4B.

For some applications, locking mechanism 260 comprises a shaft 262 and two or more attachment members 264 coupled to stent-graft 220. Shaft 262 passes through attachment members 264 when locking mechanism 260 is in the locked state, and does not pass through the attachment members when the locking mechanism is in the released state. For some applications, the locking mechanism transitions from the locked state to the released state in response to translation of the shaft, such as longitudinal translation. The shaft may be disposed either within the lumen of stent-graft 220, as shown in FIG. 4A, or outside the lumen, such as shown in FIG. 5A, mutatis mutandis.

For some applications, attachment members 264 are coupled to respective structural stent elements 228 of circumferential band 229. For some applications, structural stent elements 228 of circumferential band 229 are arranged in serpentine sections 268, each of which comprises two struts 270 connected at a respective one of peaks 232. Two attachment members 264 are coupled to circumferentially non-adjacent ones of the serpentine sections. Alternatively or additionally, for some applications, locking mechanism 260 further comprises two or more elongated coupling elements 266, which respectively couple attachment members 264 to structural stent elements 228. For some applications, each of coupling elements 266 is coupled to a structural stent element within a distance of a respective peak, which distance equals 30% of a diameter of body 222 in its first radially-expanded state. For example, the distance may equal zero, i.e., each of coupling elements 266 may be coupled to a respective peak, as shown in FIG. 4A. For other applications, coupling elements 266 are coupled to the structural stent elements at respective sites thereof other than peaks 232, such as to respective struts 270 (this configuration is not shown in FIG. 4A, but is shown in FIG. 5A, described hereinbelow). The coupling elements may be disposed either radially outside or radially inside fluid flow guide 226.

For some applications, stent-graft 220 comprises circumferential expansion element 30, described hereinabove with reference to FIGS. 1A-B and 2A-B. Alternatively or additionally, for some applications, stent-graft 220 comprises circumferential expansion prevention element 130, described hereinabove with reference to FIGS. 3A-B.

Reference is now made to FIGS. 5A-B, which are schematic illustrations of an endovascular stent-graft system 310, in accordance with an application of the present invention. Except for differences described below, stent-graft system 310 is generally similar to stent-graft system 210, described hereinabove with reference to FIGS. 4A-B, and incorporates some or all of the features thereof. Stent-graft system 310 comprises a stent-graft 320, which comprises generally tubular body 222. Body 222 is configured to assume (a) a radially-compressed delivery state, typically when the body is initially positioned in a delivery catheter, and (b) at least first and second radially-expanded deployment states. Body 222 typically assumes the first radially-expanded deployment state upon deployment from the delivery catheter, and the second radially-expanded delivery state after deployment, typically during a minimally-invasive secondary intervention procedure. FIG. 5A shows the stent-graft with body 222 in its first radially-expanded deployment state, and FIG. 5B shows the stent-graft with body 222 in its second radially-expanded deployment state.

For some applications, self-expandable flexible structural stent elements 228 of stent member 224 are shaped so to define at least one circumferential band 229 at expanding portion 223, such as exactly one circumferential band 229 or a plurality of circumferential bands 229. Circumferential band 229 is shaped so as to define a plurality of peaks 232 directed in a first longitudinal direction, alternating with a plurality of troughs 234 directed in a second longitudinal direction opposite the first longitudinal direction. Circumferential band 229 may be serpentine-shaped. Typically, stent member 224 is shaped so as to further define one or more additional circumferential bands 229 at respective longitudinal locations other than expanding portion 23, as shown in FIGS. 5A-2B.

For some applications, stent-graft system 310 further comprises locking mechanism 260, described hereinabove with reference to FIGS. 4A-B. For applications in which locking mechanism comprises shaft 262, the shaft may be disposed either radially outside the lumen of stent-graft 320, as shown in FIG. 5A, or radially inside the lumen, such as shown in FIG. 4A, mutatis mutandis.

For some applications, attachment members 264 are coupled to respective structural stent elements 228 of circumferential band 229. For some applications, structural stent elements 228 of circumferential band 229 are arranged in serpentine sections 268, each of which comprises two struts 270 connected at a respective one of peaks 232. Two attachment members 264 are coupled to circumferentially non-adjacent ones of the serpentine sections. Alternatively or additionally, coupling elements 266 are coupled to the structural stent elements at respective sites thereof other than peaks 232, such as to respective struts 270. The coupling elements may be disposed either radially outside or radially inside fluid flow guide 226. Alternatively, for some applications, such as those described in the following two paragraphs, stent-graft system 310 does not comprise locking mechanism 260.

For some applications, serpentine sections 268 of circumferential band 229 include at least one generally non-elastic serpentine section 280. Struts 270 of this serpentine section are generally non-elastic. Alternatively or additionally, these struts are substantially less elastic than the other structural stent elements. For example, an angular segment of expanding portion 223 that comprises non-elastic serpentine section 280 may expand and contract at least 30% less, such as at least 50% less, e.g., at least 67% less, per unit circumferential arc angle than an angular segment of expanding portion 223 that does not comprise non-elastic serpentine section 280, as body 222 cycles between being internally pressurized by (a) fluid having a pressure of 80 mmHg, typically by blood during diastole in an adult human, and (b) fluid having a pressure of 120 mmHg, typically by blood during systole in an adult human. For example, the struts of non-elastic serpentine section 280 may comprise non-elastic stainless steel, or a cobalt-chromium alloy. For some applications, expanding portion 223 comprises a plurality of circumferential bands 229 that include respective non-elastic serpentine sections 280. For some applications, a resistance of fluid flow guide 226 to lateral expansion is less than 70%, e.g., less than 30%, of a resistance of non-elastic serpentine section 280 to circumferential expansion.

Struts 270 of serpentine section 280 are closer together when tubular body 222 is in the first radially-expanded deployment state than when tubular body 222 is in second first radially-expanded deployment state. Optionally, struts 270 of serpentine section 280 are generally parallel to each other (e.g., define an angle of less than 30 degrees) when tubular body 222 is in the first radially-expanded deployment state. Stent member 224 is configured such that application of a force thereto, which is insufficient to cause plastic deformation of self-expandable flexible structural stent elements 228 and is sufficient to cause plastic deformation of struts 270 of serpentine section 280, transitions tubular body 222 from the first radially-expanded deployment state, as shown in FIG. 5A, to the second radially-expanded deployment state, as shown in FIG. 5B, thereby increasing a greatest internal perimeter of expanding portion 223, from a greatest internal perimeter P5 (labeled in FIG. 4A) to a greatest internal perimeter P6 (labeled in FIG. 4B). Because of their plastic deformation, struts 270 of serpentine section 280 retain their increased distance from each other even after the force is no longer applied.

For some applications, stent-graft 320 comprises circumferential expansion element 30, described hereinabove with reference to FIGS. 1A-B and 2A-B. Alternatively or additionally, for some applications, stent-graft 320 comprises circumferential expansion prevention element 130, described hereinabove with reference to FIGS. 3A-B.

Reference is now made to FIGS. 6A-B, which are schematic illustrations of an endovascular stent-graft system 410, in accordance with an application of the present invention. Except for differences described below, stent-graft system 410 is similar in some respects to the other stent-graft systems described hereinabove, and incorporates some or all of the features thereof. Stent-graft system 410 comprises a stent-graft 420, which comprises generally tubular body 422. Body 422 is configured to assume (a) a radially-compressed delivery state, typically when the body is initially positioned in a delivery catheter, and (b) at least first and second radially-expanded deployment states. Body 422 typically assumes the first radially-expanded deployment state upon deployment from the delivery catheter, and the second radially-expanded delivery state after deployment, typically during a minimally-invasive secondary intervention procedure. FIG. 6A shows the stent-graft with body 422 in its first radially-expanded deployment state, and FIG. 6B shows the stent-graft with body 422 in its second radially-expanded deployment state.

Body 422 is shaped so as to define a stepwise expanding portion 423, a greatest internal perimeter of which increases as body 422 transitions from the first radially-expanded delivery state to the second radially-expanded delivery state. For some applications, expanding portion 423 is disposed at a longitudinal end 425 of body 422, as shown in FIGS. 6A-B. For example, all of expanding portion 423 may be disposed with a distance of longitudinal end 425, measured along an axis of body 422, which distance is less than 30%, such as less than 25%, of an axial length of body 422. Alternatively or additionally, the distance is less than 120%, such as less than 80%, of an average diameter of the expanding portion when body 422 is in the first radially-expanded state. For other applications, the expanding portion is disposed elsewhere along stent-graft 420.

Body 422 comprises a stent member 424, and, typically, a generally tubular fluid flow guide 426. The fluid flow guide and the stent member are attached to each other, such as by suturing or stitching. The fluid flow guide is configured to accommodate the increase in the greatest internal perimeter of expanding portion 423, as described hereinbelow. The stent member may be attached to an internal and/or an external surface of the fluid flow guide.

Stent member 424 comprises a plurality of self-expandable flexible structural stent elements 428, which are either indirectly connected to one another by the fluid flow guide (as shown), and/or interconnected with one another (configuration not shown). Optionally, a portion of structural stent elements 428 may be attached (e.g., sutured) to the internal surface of the fluid flow guide, and another portion to the external surface of the fluid flow guide. Structural stent elements 428 comprise a self-expanding material, such as a self-expanding metal, such that body 422 is self-expandable. Typically, structural stent elements 428 comprise one or more metallic alloys, such as one or more superelastic metal alloys, a shape memory metallic alloy, and/or Nitinol. Typically, stent-graft 420 is configured to self-expand from the delivery state to the first radially-expanded deployment state. For example, stent member 424 may be heat-set to cause stent-graft 420 to self-expand from the delivery state to the first radially-expanded deployment state.

For some applications, flexible structural stent elements 428 of stent member 424 are shaped so to define at least one circumferential band 429 at expanding portion 423. Circumferential band 429 is shaped so as to define a plurality of peaks 432 directed in a first longitudinal direction, alternating with a plurality of troughs 434 directed in a second longitudinal direction opposite the first longitudinal direction. Circumferential band 429 may be serpentine-shaped. Typically, stent member 424 is shaped so as to further define one or more additional circumferential bands 429 at respective longitudinal locations other than expanding portion 423, as shown in FIGS. 6A-B.

Stent member 424 further comprises one or more circumferential expansion elements 430, which are arranged around expanding portion 423. Typically, circumferential expansion elements 430 are generally non-elastic. Alternatively or additionally, circumferential expansion elements 430 are substantially less elastic than structural stent elements 428. For example, an angular segment of expanding portion 423 that comprises one of circumferential expansion elements 430 may expand and contract at least 30% less, such as at least 50% less, e.g., at least 67% less, per unit circumferential arc angle than an angular segment of expanding portion 423 that does not comprise any of circumferential expansion elements 430, as body 422 cycles between being internally pressurized by (a) fluid having a pressure of 80 mmHg, typically by blood during diastole in an adult human, and (b) fluid having a pressure of 120 mmHg, typically by blood during systole in an adult human. For example, circumferential expansion elements 430 may comprise non-elastic stainless steel, or a cobalt-chromium alloy. For some applications, a resistance of fluid flow guide 426 to lateral expansion is less than 70%, e.g., less than 30%, of a resistance of each of circumferential expansion elements 430 to circumferential expansion.

For some applications, as shown in FIGS. 6A-B, circumferential expansion elements 430 are directly attached to fluid flow guide 426, separately from structural stent elements 428. For example, the circumferential expansion elements may be sutured to the fluid flow guide (such as in applications in which the fluid flow guide comprises polyester), or encapsulated within the fluid flow guide (such as in applications in which the fluid flow guide comprises ePTFE). For other applications, circumferential expansion elements 430 are coupled to structural stent elements 428, so as to be indirectly attached to fluid flow guide 426 (configuration not shown).

For some applications, circumferential expansion elements 430 are positioned alongside respective structural stent elements 428 near peaks 432 and/or troughs 434 of circumferential band 429 of expanding portion 423. For example, the circumferential expansion elements may be positioned within respective curvatures of peaks 432 (as shown in FIGS. 6A-B) and/or troughs 434 (configuration not shown), or outside the curvatures of the peaks and/or troughs (configuration not shown). Circumferential expansion elements 430 typically are shaped similarly to the portions of structural stent elements 428 alongside which they are positioned. For some applications, circumferential expansion elements 430 are additionally positioned alongside respective structural stent elements 428 near peaks 432 and/or troughs 434 of one or more additional circumferential bands 429 positioned along expanding portion 423. For example, in the configuration shown in FIGS. 6A-B, circumferential expansion elements 430 are positioned alongside respective structural stent elements 428 near peaks 432 of the two circumferential bands of the expanding portion.

For some applications, circumferential expansion elements 430 have a shape selected from the group of shapes consisting of: a U-shape, a V-shape, a W-shape, and an undulating shape, at least when tubular body 422 is in the first radially-expanded deployment state. Circumferential expansion elements 430 may be disposed either radially outside fluid flow guide 426, as shown in FIGS. 6A-B, or radially inside fluid flow guide 426.

Fluid flow guide 426 comprises a graft material, i.e., at least one biologically-compatible substantially blood-impervious flexible sheet. The flexible sheet may comprise, for example, a polyester, a polyethylene (e.g., a poly-ethylene-terephthalate), a polymeric film material (such as a fluoropolymer, e.g., polytetrafluoroethylene), a polymeric textile material (e.g., woven polyethylene terephthalate (PET)), natural tissue graft (e.g., saphenous vein or collagen), Polytetrafluoroethylene (PTFE), ePTFE, Dacron, or a combination of two or more of these materials. The graft material optionally is woven. For some applications, the graft material of fluid flow guide 426 is generally non- or minimally-elastic.

Stent member 424 is configured such that application of a force thereto, which is insufficient to cause plastic deformation of self-expandable flexible structural stent elements 428 and is sufficient to cause plastic deformation of circumferential expansion elements 430, causes plastic deformation of and an increase in respective circumferential lengths of circumferential expansion elements 430, from a first length, as shown in FIG. 6A, to a second length, as shown in FIG. 6B. This increase in length transitions tubular body 422 from the first radially-expanded deployment state, as shown in FIG. 6A, to the second radially-expanded deployment state, as shown in FIG. 6B, thereby increasing a greatest internal perimeter of expanding portion 423, from a first greatest internal perimeter to a second greatest internal perimeter. Because of the plastic deformation, circumferential expansion elements 430 retain their increased lengths even after the force is no longer applied. For applications in which a plurality of circumferential expansion elements 430 is provided, the circumferential expansion is generally distributed over the plurality of elements.

Typically, circumferential expansion element 430, or, for applications in which stent member 424 comprises a plurality of circumferential expansion elements 430, circumferential expansion elements 430 collectively circumscribe an aggregate angle of at least 20 degrees, when tubular body 422 is in the first radially-expanded deployment state, as shown in FIGS. 6A. Typically, each of circumferential expansion elements 430 circumscribes an angle of at least 3 degrees, such as at least 5 degrees, when tubular body 422 is in the first radially-expanded deployment state, as shown in FIGS. 6A. For example, the angle may be at least 40 degrees, such as at least 90 degrees. For some applications, when tubular body 422 is the second radially-expanded deployment state, circumferential expansion element 430 circumscribes an angle that is capable of attaining a value that is at least 30% greater than when tubular body 422 is the first radially-expanded deployment state.

As mentioned above, fluid flow guide 426 is configured to accommodate the increase in the greatest internal perimeter of expanding portion 423. For some applications, in order to provide such accommodation, when tubular body 422 is in the first radially-expanded deployment state, fluid flow guide 426 is shaped so as to define one or more folds 440 in a vicinity of circumferential expansion element 430, such as shown in FIG. 6A. For some applications, such as shown in FIG. 6A, when tubular body 422 is in the first radially-expanded deployment state, the one or more folds are disposed radially outside stent member 424.

Alternatively, for some applications, in order to provide such accommodation, at least a portion of fluid flow guide 426 in a vicinity of circumferential expansion elements 430 comprises a stretchable fabric (this configuration is not shown in FIGS. 6A-B, but is similar to the configuration shown in FIG. 3A, described hereinabove). For example, the stretchable fabric may comprise expanded polytetrafluoroethylene (ETFE). For some applications, fluid flow guide 426, other than the portion in the vicinity of circumferential expansion element 430, comprises a fabric that is less elastic than the stretchable fabric. For example, the fabric of an angular segment of expanding portion 423 that comprises one of circumferential expansion elements 430 may expand and contract at least 30% less, such as at least 50% less, e.g., at least 67% less, per unit circumferential arc angle than the fabric of an angular segment of expanding portion 423 that does not comprise any of circumferential expansion elements 430, as body 422 cycles between being internally pressurized by (a) fluid having a pressure of 80 mmHg, typically by blood during diastole in an adult human, and (b) fluid having a pressure of 120 mmHg, typically by blood during systole in an adult human.

For some applications, stent-graft 420 further comprises at least circumferential expansion element 30, described hereinabove with reference to FIGS. 1A-B and 2A-B. Alternatively or additionally, for some applications, stent-graft 420 comprises at least one circumferential expansion prevention element 130, described hereinabove with reference to FIGS. 3A-B. Further alternatively or additionally, for some applications, stent-graft 420 comprises one or more folds 230, described hereinabove with reference to FIGS. 4A-B. Further alternatively or additionally, for some applications, stent-graft 420 comprises at least one non-elastic serpentine section 280, described hereinabove with reference to FIGS. 5A-B.

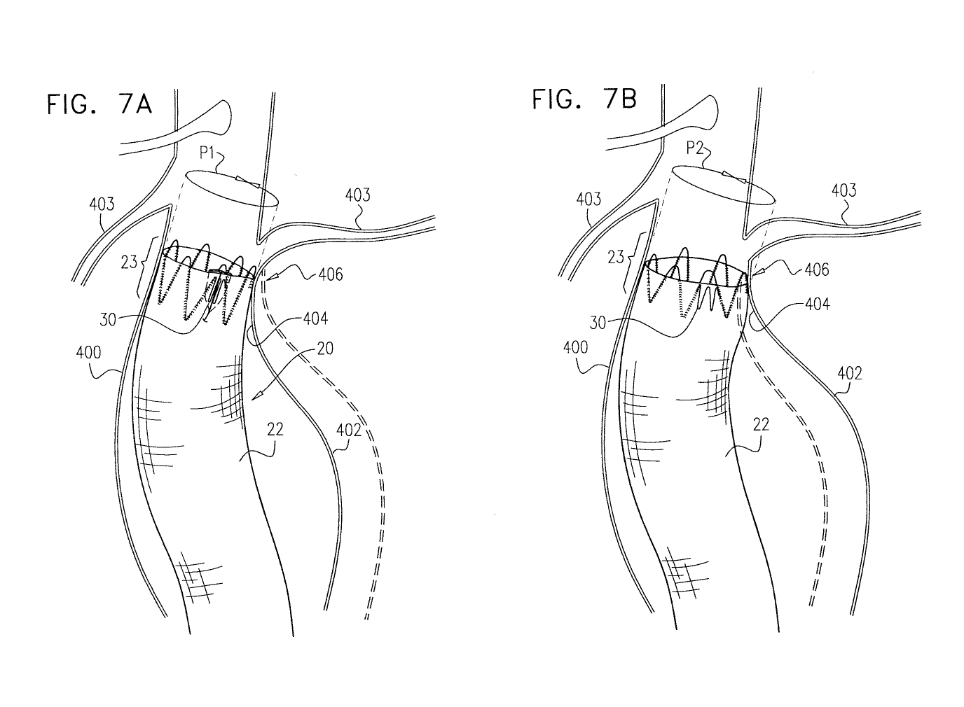

Reference is now made to FIGS. 7A-B, which are schematic illustrations of an exemplary method for deploying stent-graft 20, described hereinabove with reference to FIGS. 1A-B and 2A-B, in accordance with an application of the present invention. In this exemplary method, stent-graft 20 is configured to be implanted in a main blood vessel having an aneurysm and/or a dissection, such as a descending abdominal aorta 400 (which may have an aneurysm 402, typically below renal arteries 403, as shown).

During a primary intervention procedure, a surgeon or interventionalist transvascularly introduces stent-graft 20 into the blood vessel while tubular body 22 of the stent-graft is in the radially-compressed delivery state. Thereafter, the surgeon or interventionalist transitions the tubular body to the first radially-expanded deployment state in the blood vessel, in which state expanding portion 23 has first greatest internal perimeter P1 and forms a blood-tight seal with a wall 404 of the blood vessel at a neck 406 of aneurysm 402 and/or the dissection. The initial implantation procedure is complete, as shown in FIG. 7A.

Over time (typically over a few several years), neck 406 often progressively dilates, such as because of progressive expansion of the aneurysm sac. Such dilation of the neck may compromise the seal between expanding portion 23 of the stent-graft and the wall of neck 406, resulting in type I endoleak. In response to detecting such dilation and/or endoleak (typically at least one month, such as at least a few years, after initial implantation and deployment of the stent-graft), a surgeon or interventionalist, during a minimally-invasive secondary intervention procedure, transitions tubular body 22 to a second radially-expanded deployment state in the blood vessel, as shown in FIG. 7B. In the second radially-expanded deployment state, expanding portion 23 has a second greatest internal perimeter P2, which is greater than first greatest perimeter P1. Typically, the minimally-invasive secondary intervention procedure is performed transvascularly and most likely transcutaneously.

For some applications, in order to transition tubular body 22 to the second radially-expanded deployment state in the blood vessel, the surgeon or interventionalist transvascularly introduces a balloon into tubular body 22, and inflates the balloon. The balloon applies a force to stent member 24 to cause plastic deformation of circumferential expansion element 30, as described hereinabove with reference to FIGS. 1A-B and 2A-B. Optionally, a bare metal stent is further provided, initially disposed over a delivery balloon. This bare metal stent, typically crimped over the balloon, is advanced within tubular body 22, while the bare metal stent is in a radially-compressed state and the balloon is deflated. The balloon is then inflated to transition the bare metal stent to a radially-expanded state, in which the bare metal stent has a greater diameter than that of stent-graft 20 when in the first radially-expanded deployment state. This expansion of the bare metal stent thus transitions stent-graft 20 to the larger second radially-expanded deployment state. The bare metal stent is typically left in place in stent-graft 20. For some applications, the bare metal stent is plastically deformable (e.g., comprises stainless steel), while for other applications the bare metal stent is superelastic (e.g., comprises Nitinol).

For some applications, tubular body 22 is configured to undergo one or more additional transitions to one or more additional radially-expanded deployment states in which expanding portion 23 of stent-graft 20 has respective even greater radially-expanded internal perimeters. Such additional transitions may be effected if neck 406 of aneurysm 402 and/or the dissection further dilates after body 22 has transitioned to the second radially-expanded deployment state, or if the transition to the second radially-expanded deployment state is insufficient to resolve the initial endoleak. For example, body 22 may be configured to assume a third radially-expanded deployment state, in which expanding portion 23 of stent-graft 20 has a third greatest internal perimeter, which is greater than second greatest internal perimeter P2, described hereinabove with reference to FIG. 2B. A surgeon or interventionalist transitions tubular body 22 to the additional radially-expanded deployment states during respective subsequent minimally-invasive secondary intervention procedures, or during the first secondary intervention procedure if necessary to resolve the endoleak. For example, a plurality of balloons may be provided that have respective different volumes when inflated.

For some applications, in order to enable such additional transitions, stent member 24 further comprises one or more additional circumferential expansion elements 30 at additional respective circumferential locations. Alternatively or additionally, for some applications, stent member 24 comprises a plurality of circumferential expansion elements 30 at a plurality of circumferential bands 29, respectively. Alternatively or additionally, in order to enable such additional transitions, circumferential expansion element 30 is configured to enable more than one change in circumferential length L thereof (labeled in FIGS. 1A-B and 2A-B).

Reference is now made to FIGS. 8A-B, which are schematic illustrations of an exemplary method for deploying stent-graft 220, described hereinabove with reference to FIGS. 4A-B, in accordance with an application of the present invention. In this exemplary method, stent-graft 220 is configured to be implanted in a main blood vessel having an aneurysm and/or a dissection, such as descending abdominal aorta 400 (which may have aneurysm 402, typically below renal arteries 403, as shown).

During a primary intervention procedure, a surgeon or interventionalist transvascularly introduces stent-graft 220 into the blood vessel while tubular body 222 of the stent-graft is in the radially-compressed delivery state. Thereafter, the surgeon or interventionalist transitions the tubular body to a first radially-expanded deployment state in the blood vessel, in which state expanding portion 223 has first greatest internal perimeter P5 and forms a blood-tight seal with wall 404 of the blood vessel at neck 406 of aneurysm 402 and/or the dissection. The initial implantation procedure is complete, as shown in FIG. 8A.

As mentioned above, over time (typically over several months to several years), neck 406 often progressively dilates, such as because of progressive expansion of the aneurysm sac. Such dilation of the neck may compromise the seal between expanding portion 223 of the stent-graft and the wall of neck 406, resulting in type I endoleak. In response to detecting such dilation and/or endoleak (typically at least one month, such as at least a few years, after implantation of the stent-graft), a surgeon or interventionalist, during a minimally-invasive secondary intervention procedure, transitions tubular body 222 to a second radially-expanded deployment state in the blood vessel, as shown in FIG. 8B. In the second radially-expanded deployment state, expanding portion 223 has a second greatest internal perimeter P6, which is greater than first greatest perimeter P5. Typically, the minimally-invasive secondary intervention procedure is performed transvascularly.

For applications in which stent-graft system 210 comprises locking mechanism 260, in order to transition tubular body 222 to the second radially-expanded deployment state in the blood vessel, the surgeon or interventionalist transitions the locking mechanism from the locked state to the unlocked state, which allows tubular body 222 to transition to the second radially-expanded deployment state. For applications in which locking mechanism 260 comprises shaft 262, the surgeon or interventionalist transvascularly translates the shaft in order to unlock locking mechanism 260.

For some applications, tubular body 222 is configured to undergo one or more additional transitions to one or more additional radially-expanded deployment states in which expanding portion 223 of stent-graft 220 has respective even greater radially-expanded internal perimeters. Such additional transitions may be effected if neck 406 of aneurysm 402 and/or the dissection further dilates after body 222 has transitioned to the second radially-expanded deployment state, or if the transition to the second radially-expanded deployment state is insufficient to resolve the initial endoleak. For example, body 222 may be configured to assume a third radially-expanded deployment state, in which expanding portion 223 of stent-graft 220 has a third greatest internal perimeter, which is greater than second greatest internal perimeter P6, described hereinabove with reference to FIG. 4B. A surgeon or interventionalist transitions tubular body 222 to the additional radially-expanded deployment states during respective subsequent minimally-invasive secondary intervention procedures, or during the first secondary intervention procedure if necessary to resolve the endoleak.

For some applications, in order to enable such additional transitions, stent member 24 further comprises one or more additional folds 230 and corresponding locking mechanisms 260 at additional respective circumferential locations, as described hereinabove with reference to FIGS. 4A-B. For applications in which the locking mechanisms comprise respective shafts 262, the surgeon or interventionalist transvascularly translates the shafts in respective post-implantation minimally-invasive secondary intervention procedures in order to unlock the respective locking mechanisms.

In order to deploy stent-graft 320, described hereinabove with reference to FIGS. 5A-B, the deployment techniques may be used that are described hereinabove with reference to FIGS. 7A-B and/or 8A-B, depending on the configuration of stent-graft 320. For configurations in which stent-graft 320 comprises generally non-elastic serpentine section 280, the techniques described hereinabove with reference to FIGS. 7A-B may be used. Alternatively or additionally, for configurations in which stent-graft system 310 comprises locking mechanism 260, the techniques described hereinabove with reference to FIGS. 8A-B may be used.

Stent-graft 120, described hereinabove with reference to FIGS. 3A-B, may be deployed using techniques similar to those described hereinabove with reference to FIGS. 7A-B. For some applications, a balloon is expanded within the lumen of stent-graft 120 to apply a force to and detach and/or sever circumferential expansion prevention element 130. Alternatively, a cutting tool may be transvascularly introduced into the stent-graft, and used to cut circumferential expansion prevention element 130. For some applications, stent-graft 120 comprises a plurality of circumferential expansion prevention elements 130, located at respective circumferential locations. Detaching and/or severing elements 130 transitions tubular body 122 to transition to one or more additional radially-expanded deployment states in which expanding portion 123 of stent-graft 120 has respective even greater radially-expanded internal perimeters. In order to effect such additional transitions, the techniques described hereinabove with reference to FIGS. 7A-B may be used, mutatis mutandis.

As used in the present application, including in the claims, “tubular” means having the form of an elongated hollow object that defines a conduit therethrough. A “tubular” structure may have varied cross-sections therealong, and the cross-sections are not necessarily circular. For example, one or more of the cross-sections may be generally circular, or generally elliptical but not circular, or circular.

The scope of the present invention includes embodiments described in the following applications, which are assigned to the assignee of the present application and are incorporated herein by reference. In an embodiment, techniques and apparatus described in one or more of the following applications are combined with techniques and apparatus described herein:
PCT Application PCT/IL2008/000287, filed Mar. 5, 2008, which published as PCT Publication WO 2008/107885 to Shalev et al., and U.S. application Ser. No. 12/529,936 in the national stage thereof, which published as U.S. Patent Application Publication 2010/0063575 to Shalev et al.
U.S. Provisional Application 60/892,885, filed Mar. 5, 2007
PCT Application PCT/IL2007/001312, filed Oct. 29, 2007, which published as PCT Publication WO/2008/053469 to Shalev, and U.S. application Ser. No. 12/447,684 in the national stage thereof, which published as US Patent Application Publication 2010/0070019 to Shalev
U.S. Provisional Application 60/991,726, filed Dec. 2, 2007
PCT Application PCT/IL2008/001621, filed Dec. 15, 2008, which published as PCT Publication WO 2009/078010, and U.S. application Ser. No. 12/808,037 in the national stage thereof, which published as U.S. Patent Application Publication 2010/0292774
U.S. Provisional Application 61/219,758, filed Jun. 23, 2009
U.S. Provisional Application 61/221,074, filed Jun. 28, 2009
PCT Application PCT/IB2010/052861, filed Jun. 23, 2010, which published as PCT Publication WO 2010/150208, and U.S. application Ser. No. 13/380,278 in the national stage thereof, which published as US Patent Application Publication 2012/0150274
PCT Application PCT/IL2010/000549, filed Jul. 8, 2010, which published as PCT Publication WO 2011/004374
PCT Application PCT/IL2010/000564, filed Jul. 14, 2010, which published as PCT Publication WO 2011/007354, and U.S. application Ser. No. 13/384,075 in the national stage thereof, which published as US Patent Application Publication 2012/0179236
PCT Application PCT/IL2010/000917, filed Nov. 4, 2010, which published as PCT Publication WO 2011/055364
PCT Application PCT/IL2010/000999, filed Nov. 30, 2010, which published as PCT Publication WO 2011/064782
PCT Application PCT/IL2010/001018, filed Dec. 2, 2010, which published as PCT Publication WO 2011/067764
PCT Application PCT/IL2010/001037, filed Dec. 8, 2010, which published as PCT Publication WO 2011/070576
PCT Application PCT/IL2010/001087, filed Dec. 27, 2010, which published as PCT Publication WO 2011/080738
PCT Application PCT/IL2011/000135, filed Feb. 8, 2011, which published as PCT Publication WO 2011/095979
PCT Application PCT/IL2011/000801, filed Oct. 10, 2011, which published as PCT Publication WO 2012/049679
U.S. Application 13/031,871, filed Feb. 22, 2011, which published as US Patent Application Publication 2011/0208289
U.S. Provisional Application 61/496,613, filed Jun. 14, 2011
U.S. Provisional Application 61/505,132, filed Jul. 7, 2011
U.S. Provisional Application 61/529,931, filed Sep. 1, 2011

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1-29. (canceled)

30. Apparatus comprising an endovascular stent-graft, which comprises a generally tubular body, which tubular body (a) is configured to assume a radially-compressed delivery state and at least first and second radially-expanded deployment states, (b) is shaped so as to define a stepwise expanding portion, and (c) comprises a stent member, which comprises:
a plurality of self-expandable flexible structural stent elements; and
at least one circumferential expansion element,
wherein the stent member is configured such that application of a force thereto, which is insufficient to cause plastic deformation of the self-expandable flexible structural stent elements and is sufficient to cause plastic deformation of the circumferential expansion element, causes an increase in a circumferential length of the circumferential expansion element, thereby transitioning the tubular body from the first radially-expanded deployment state to the second radially-expanded deployment state, thereby increasing a greatest internal perimeter of the expanding portion.

31. The apparatus according to claim 30, wherein the circumferential expansion element circumscribes an angle of at least 3 degrees, when the tubular body is in the first radially-expanded deployment state.

32. (canceled)

33. The apparatus according to claim 30, wherein the circumferential expansion element is coupled to at least two of the self-expandable flexible structural stent elements of the expanding portion of the tubular body.

34. (canceled)

35. The apparatus according to claim 30 wherein the self-expandable flexible structural stent elements of the stent member are shaped so to define at least one circumferential band at the expanding portion, which band is shaped so as to define a plurality of peaks directed in a first longitudinal direction, alternating with a plurality of troughs directed in a second longitudinal direction opposite the first longitudinal direction.

36. The apparatus according to claim 35, wherein the at least one circumferential expansion element is positioned alongside one of the self-expandable flexible structural stent elements near an element selected from the group consisting of: one of the peaks and one of the troughs.

37. (canceled)

38. The apparatus according to claim 30 wherein the tubular body further comprises a generally tubular fluid flow guide, which (a) comprises a graft material, (b) is attached to the stent member, and (c) is configured to accommodate the increasing of the greatest internal perimeter of the expanding portion.

39. The apparatus according to claim 38, wherein the at least one circumferential expansion element is attached to the fluid flow guide.

40. The apparatus according to claim 38, wherein, when the tubular body is in the first radially-expanded deployment state, the fluid flow guide is shaped so as to define one or more folds in a vicinity of the circumferential expansion element, so as to accommodate the increasing of the greatest internal perimeter of the expanding portion.

41. The apparatus according to claim 40, wherein, when the tubular body is in the first radially-expanded deployment state, the one or more folds are disposed radially outside the stent member.

42. The apparatus according to claim 38, wherein at least a portion of the fluid flow guide in a vicinity of the circumferential expansion element comprises a stretchable fabric, so as to accommodate the increasing of the greatest internal perimeter of the expanding portion.

43. (canceled)

44. The apparatus according to claim 38, wherein a resistance of the fluid flow guide to lateral expansion is less than 70% of a resistance of the circumferential expansion element to lateral expansion, when the tubular body is in the second radially-expanded deployment state.

45. (canceled)

46. The apparatus according to claim 30 wherein the circumferential expansion element has a shape selected from the group of shapes consisting of: a U-shape, a V-shape, a W-shape, and an undulating shape, at least when the tubular body is in the first radially-expanded deployment state.

47-48. (canceled)

49. The apparatus according to claim 30 wherein the circumferential expansion element comprises non-elastic stainless steel.

50. The apparatus according to claim 30 wherein the circumferential expansion element is generally non-elastic.

51. (canceled)

52. The apparatus according to claim 30 wherein the circumferential expansion element comprises a cobalt-chromium alloy.

53-65. (canceled)

66. A method comprising:
providing an endovascular stent-graft, which includes a generally tubular body, which (a) is shaped so as to define a stepwise expanding portion, and (b) includes a self-expandable flexible stent member, and a generally tubular fluid flow guide, which includes a graft material and is attached to the stent member;
during a minimally-invasive primary intervention procedure, transvascularly introducing the stent-graft into a blood vessel of a human subject while the tubular body of the stent-graft is in a radially-compressed delivery state, and, thereafter, transitioning the tubular body to a first radially-expanded deployment state in the blood vessel, in which state the expanding portion has a first greatest internal perimeter and forms a blood-tight seal with a wall of the blood vessel; and
thereafter, during a minimally-invasive secondary intervention procedure separate from the primary intervention procedure, transitioning the tubular body to a second radially-expanded deployment state in the blood vessel, in which state the expanding portion has a second greatest internal perimeter and forms a blood-tight seal with the wall of the blood vessel, which second greatest internal perimeter is greater than the first greatest internal perimeter.

67. The method according to claim 66, transitioning the tubular body to the second radially-expanded deployment state in the blood vessel comprises performing the secondary intervention procedure at least one month after performing the primary intervention procedure.

68. The method according to claim 66, wherein the minimally-invasive secondary intervention procedure is a transvascular secondary intervention procedure, and wherein transitioning the tubular body to the second radially-expanded deployment state comprises transitioning the tubular body to the second radially-expanded deployment state during the transvascular secondary intervention procedure.

69. (canceled)

70. The method according to claim 66, further comprising, after the minimally-invasive secondary intervention procedure, during a minimally-invasive tertiary intervention procedure separate from the primary and the secondary intervention procedures, transitioning the tubular body to a third radially-expanded deployment state in the blood vessel, in which state the expanding portion has a third greatest internal perimeter and forms a blood-tight seal with the wall of the blood vessel, which third greatest internal perimeter is greater than the second greatest internal perimeter.

71. The method according to claim 66, further comprising, after transitioning the tubular body to the first radially-expanded deployment state, detecting type I endoleak, and wherein transitioning the tubular body to the second radially-expanded deployment state comprises transitioning the tubular body to the second radially-expanded deployment state in response to detecting the type I endoleak.

72. The method according to claim 66,
further comprising identifying that the blood vessel has an aneurysm,
wherein transitioning the tubular body to the first radially-expanded deployment state comprises transitioning the tubular body to the first radially-expanded deployment state so that the expanding portion forms the blood-tight seal with the wall of the blood vessel at a neck of the aneurysm, and
wherein transitioning the tubular body to the second radially-expanded deployment state comprises transitioning the tubular body to the second radially-expanded deployment state so that the expanding portion forms the blood-tight seal with the wall of the blood vessel at the neck of the aneurysm.

73. The method according to claim 66, wherein transitioning the tubular body to the second radially-expanded deployment state comprises transitioning the tubular body to the second radially-expanded deployment state such that the second greatest internal perimeter of the expanding portion is at least 10% greater than the first greatest internal perimeter of the expanding portion.

74-103. (canceled)

104. The method according to claim 66,
wherein providing the endovascular stent-graft comprises providing the endovascular stent-graft in which the tubular body further includes a stent member, which includes a plurality of self-expandable flexible structural stent elements, and at least one circumferential expansion element, and
wherein transitioning the tubular body to a second radially-expanded deployment state comprises causing an increase in a circumferential length of the circumferential expansion element, by applying a force to the stent member, which force is insufficient to cause plastic deformation of the self-expandable flexible structural stent elements and is sufficient to cause plastic deformation of the circumferential expansion element.

105. The method according to claim 104, wherein providing the endovascular stent-graft comprises providing the endovascular stent-graft in which the circumferential expansion element circumscribes an angle of at least 3 degrees, when the tubular body is in the first radially-expanded deployment state.

106. (canceled)

107. The method according to claim 104, wherein providing the endovascular stent-graft comprises providing the endovascular stent-graft in which the circumferential expansion element is coupled to at least two of the self-expandable flexible structural stent elements of the expanding portion of the tubular body.

108-111. (canceled)

112. The method according to claim 104, wherein providing the endovascular stent-graft comprises providing the endovascular stent-graft in which at least a portion of the fluid flow guide in a vicinity of the circumferential expansion element includes a stretchable fabric, so as to accommodate the increasing of the greatest internal perimeter of the expanding portion.

113. The method according to claim 104, wherein providing the endovascular stent-graft comprises providing the endovascular stent-graft in which the circumferential expansion element has a shape selected from the group of shapes consisting of: a U-shape, a V-shape, a W-shape, and an undulating shape, at least when the tubular body is in the first radially-expanded deployment state.

114-115. (canceled)

116. The method according to claim 104, wherein providing the endovascular stent-graft comprises providing the endovascular stent-graft in which the circumferential expansion element includes non-elastic stainless steel.

117. The method according to claim 104, wherein providing the endovascular stent-graft comprises providing the endovascular stent-graft in which the circumferential expansion element is generally non-elastic.

118. (canceled)

119. The method according to claim 104, wherein providing the endovascular stent-graft comprises providing the endovascular stent-graft in which the circumferential expansion element includes a cobalt-chromium alloy.

120-125. (canceled)
